SEQUENCE LISTING (49 sequences)

SEQ ID NO: 1 - DNA, 15 bp, Artificial Sequence
  Synthetic
gggagaccaggaatt

SEQ ID NO: 2 - DNA, 15 bp, Artificial Sequence
  Synthetic
gggagaccgggaatt

SEQ ID NO: 3 - DNA, 15 bp, Artificial Sequence
  Synthetic
gggagacccggaatt

SEQ ID NO: 4 - DNA, 14 bp, Artificial Sequence
  Synthetic
gggagacctggaat

SEQ ID NO: 5 - DNA, 14 bp, Artificial Sequence
  Synthetic
gggagaccacaacg

SEQ ID NO: 6 - DNA, 14 bp, Artificial Sequence
  Synthetic
gggagaccggaatt

SEQ ID NO: 7 - DNA, 15 bp, Artificial Sequence
  Synthetic
gggagaccggaattt

SEQ ID NO: 8 - DNA, 6 bp, Artificial Sequence
  Synthetic
gggaga

SEQ ID NO: 9 - DNA, 17 bp, Artificial Sequence
  Synthetic
taatacgactcactata

SEQ ID NO: 10 - DNA, 32 bp, Artificial Sequence
  Synthetic
taatacgactcactatagggagaccaggaatt

SEQ ID NO: 11 - DNA, 32 bp, Artificial Sequence
  Synthetic
taatacgactcactatagggagaccgggaatt

SEQ ID NO: 12 - DNA, 32 bp, Artificial Sequence
  Synthetic
taatacgactcactatagggagacccggaatt

SEQ ID NO: 13 - DNA, 32 bp, Artificial Sequence
  Synthetic
taatacgactcactatagggagacctggaatt

SEQ ID NO: 14 - DNA, 32 bp, Artificial Sequence
  Synthetic
taatacgactcactatagggagaccacaacgt

SEQ ID NO: 15 - DNA, 31 bp, Artificial Sequence
  Synthetic
taatacgactcactatagggagaccggaatt

SEQ ID NO: 16 - DNA, 32 bp, Artificial Sequence
  Synthetic
taatacgactcactatagggagaccggaattt

SEQ ID NO: 17 - DNA, 23 bp, Artificial Sequence
  Synthetic
taatacgactcactatagggaga

SEQ ID NO: 18 - DNA, 47 bp, Artificial Sequence
  Synthetic
tcgattcgaacttctgatagacttcgaaattaatacgactcactata

SEQ ID NO: 19 - DNA, 41 bp, Artificial Sequence
  Synthetic
aaccccttggggcctctaaacgggtcttgaggggttttttg

SEQ ID NO: 20 - DNA, 106 bp, Artificial Sequence
  Synthetic
catctgttttcttgcaagatcagctgagcaataactagcataaccccttggggcctctaaacgggtcttgaggggttttttgctgaaaggaggaactatatccgga

SEQ ID NO: 21 - DNA, 295 bp, Artificial Sequence
  Synthetic
cctagcataaccccgcggggcctcttcgggggtctcgcggggttttttgctgaaagaagcttcaaataaaacgaaaggctcagtcgaaagactgggcctttcgttttatctgttgtttgtcgctgcggccgcactcgagcaccaccaccaccaccattgagatccggctgctaacaaagcccgaaaggaagctgagttggctgctgccaccgctgagcaataactagcataaccccttggggcctctaaacgggtcttgaggggttttttgctgaaaggaggaactatatccgga

SEQ ID NO: 22 - DNA, 297 bp, Artificial Sequence
  Synthetic
cctagcataaaccccttgggttccctctttaggagtctgaggggttttttgctgaaagaagcttcaaataaaacgaaaggctcagtcgaaagactgggcctttcgttttatctgttgtttgtcgctgcggccgcactcgagcaccaccaccaccaccattgagatccggctgctaacaaagcccgaaaggaagctgagttggctgctgccaccgctgagcaataactagcataaccccttggggcctctaaacgggtcttgaggggttttttgctgaaaggaggaactatatccgga

SEQ ID NO: 23 - DNA, 185 bp, Artificial Sequence
  Synthetic
aagcttgcttaagcagaaggccatcctgacggatggcctttttgcgtttctacctagcataaccccttggggcctctaaacgggtcttgaggggttttttggccatctgttttcttgcaagatcagctgagcaataactagcataaccccttggggcctctaaacgggtcttgaggggttttttg

SEQ ID NO: 24 - DNA, 69 bp, Artificial Sequence
  Synthetic
tcaaataaaacgaaaggctcagtcgaaagactgggcctttcgttttatctgttgtttgtcgctgcggcc

SEQ ID NO: 25 - DNA, 45 bp, Artificial Sequence
  Synthetic
ttaagcagaaggccatcctgacggatggcctttttgcgtttctac

SEQ ID NO: 26 - DNA, 48 bp, Artificial Sequence
  Synthetic
ctagcataaccccttggggcctctaaacgggtcttgaggggttttttg

SEQ ID NO: 27 - DNA, 84 bp, Artificial Sequence
  Synthetic
gctgagcaataactagcataaccccttggggcctctaaacgggtcttgaggggttttttgctgaaaggaggaactatatccgga

SEQ ID NO: 28 - DNA, 61 bp, Artificial Sequence
  Synthetic
cctagcataaccccgcggggcctcttcgggggtctcgcggggttttttgctgaaagaagct

SEQ ID NO: 29 - DNA, 63 bp, Artificial Sequence
  Synthetic
cctagcataaaccccttgggttccctctttaggagtctgaggggttttttgctgaaagaagct

SEQ ID NO: 30 - DNA, 10 bp, Artificial Sequence
  Synthetic
catctgtttt

SEQ ID NO: 31 - DNA, 47 bp, Artificial Sequence; identical to SEQ ID NO: 18
  Synthetic

SEQ ID NO: 32 - DNA, 53 bp, Artificial Sequence
  Synthetic
tcgattcgaacttctgatagacttcgaaattaatacgactcactatagggaga

SEQ ID NO: 33 - DNA, 62 bp, Artificial Sequence
  Synthetic
tcgattcgaacttctgatagacttcgaaattaatacgactcactatagggagaccacaacgt

SEQ ID NO: 34 - DNA, 61 bp, Artificial Sequence
  Synthetic
tcgattcgaacttctgatagacttcgaaattaatacgactcactatagggagaccggaatt

SEQ ID NO: 35 - DNA, 62 bp, Artificial Sequence
  Synthetic
tcgattcgaacttctgatagacttcgaaattaatacgactcactatagggagaccgggaatt

SEQ ID NO: 36 - DNA, 19 bp, Artificial Sequence
  Synthetic
taatacgactcactatagg

SEQ ID NO: 37 - DNA, 30 bp, Artificial Sequence
  Synthetic
tcgattcgaacttctgatagacttcgaaat

SEQ ID NO: 38 - DNA, 15 bp, Artificial Sequence
  Synthetic
aggagaccaggaatt

SEQ ID NO: 39 - DNA, 15 bp, Artificial Sequence
  Synthetic
aggagaccgggaatt

SEQ ID NO: 40 - DNA, 15 bp, Artificial Sequence
  Synthetic
aggagacccggaatt

SEQ ID NO: 41 - DNA, 14 bp, Artificial Sequence
  Synthetic
aggagacctggaat

SEQ ID NO: 42 - DNA, 32 bp, Artificial Sequence
  Synthetic
taatacgactcactataaggagaccaggaatt

SEQ ID NO: 43 - DNA, 32 bp, Artificial Sequence
  Synthetic
taatacgactcactataaggagaccgggaatt

SEQ ID NO: 44 - DNA, 32 bp, Artificial Sequence
  Synthetic
taatacgactcactataaggagacccggaatt

SEQ ID NO: 45 - DNA, 32 bp, Artificial Sequence
  Synthetic
taatacgactcactataaggagacctggaatt

SEQ ID NO: 46 - DNA, 48 bp, Artificial Sequence
  Synthetic
tcgattcgaacttctgatagacttcgaaattaatacgactcactatag

SEQ ID NO: 47 - DNA, 49 bp, Artificial Sequence
  Synthetic
tcgattcgaacttctgatagacttcgaaattaatacgactcactatagg

SEQ ID NO: 48 - DNA, 50 bp, Artificial Sequence
  Synthetic
tcgattcgaacttctgatagacttcgaaattaatacgactcactataggg

SEQ ID NO: 49 - DNA, 53 bp, Artificial Sequence; identical to SEQ ID NO: 32
  Synthetic

(19) United States
(12) Patent Application Publication
(10) Pub. No.: US 2022/0411792 A1
(43) Pub. Date: Dec. 29, 2022

(54) NUCLEIC ACID COMPOSITIONS

(71) Applicant: GREENLIGHT BIOSCIENCES, INC., Medford, MA (US)

(72) Inventors: James Robbins ABSHIRE, Cambridge, MA (US); Himanshu DHAMANKAR, Arlington, MA (US); William FARMER, Concord, MA (US); Christopher GREGG, Melrose, MA (US)

(21) Appl. No.: 17/779,257

(22) PCT Filed: Dec. 4, 2020
(86) PCT No.: PCT/US2020/063490

§ 371 Date: May 24, 2022

Related U.S. Application Data
(60) Provisional application No. 62/944,824, filed on Dec. 6, 2019.

(51) Int. Cl. C12N 15/11; C12N 15/113; C12N 15/63

(52) U.S. Cl. CPC C12N 15/111; C12N 15/113; C12N 15/63; C12N 2310/14; C12N 2830/34; C12N 2830/36; C12N 2330/51

(57) ABSTRACT

Provided herein, in some aspects, are compositions of nucleic acids comprising an initial transcribed sequence and unique nucleic acid designs for high-yield and cost-effective production of ribonucleic acids.

### RELATED APPLICATION

This application claims the benefit under 35 U.S.C. § 119(e) of U.S. provisional application No. 62/944,824, filed Dec. 6, 2019, which is incorporated by reference herein in its entirety.

### BACKGROUND OF THE INVENTION

The availability of low-cost RNA products is essential for numerous applications spanning the agricultural and biopharmaceutical sciences. In agriculture, RNA interference (RNAi) can be used for targeted control of pests and insects that are increasingly resistant to traditional chemical pesticides and for effecting specific desired phenotypes in crops (e.g., improved shelf life, color, freshness). In biopharmaceuticals, mRNA products can be used as vaccines as well as therapeutics to treat different diseases. However, the high cost of RNA synthesis is a major hurdle in the widespread use and development of RNA products. Developing cost-effective synthesis processes for RNA products enables widespread expansion and use of these and other technologies.

### SUMMARY OF THE INVENTION

Some aspects of the present disclosure provide recombinant DNA nucleic acid construct designs for the enhanced expression of an RNA of interest. In some embodiments, a construct comprises: a first expression cassette comprising a promoter operably linked to an initial transcription sequence (ITS) upstream of a nucleotide sequence encoding a sense strand of a double-stranded RNA (dsRNA); and a second expression cassette comprising a promoter operably linked to an initial transcription sequence (ITS) upstream of a nucleotide sequence encoding an antisense strand of the dsRNA, wherein the sense strand of the dsRNA is complementary to the antisense strand of the dsRNA.

In some embodiments, either or both of the first and second expression cassette further comprise a terminator sequence downstream of the nucleotide sequence encoding a strand of the dsRNA. In some embodiments, either or both of the first and second expression cassette further comprise a restriction endonuclease recognition site. In some embodiments, either or both of the first and second expression cassette comprise a terminator sequence downstream of the nucleotide sequence encoding a strand of the dsRNA and further comprise an endonuclease recognition site.

In some embodiments, the initial transcription sequences have a length of 1-15 nucleotides. In some embodiments, the initial transcription sequences comprise the nucleotide sequence of any one of SEQ ID NOs: 1-8 or 38-41.

In some embodiments, the first expression cassette and the second expression cassette are located within a single DNA molecule and are oriented in the same direction; wherein the same DNA strand serves as the template strand during transcription for each expression cassette. In other embodiments, the first expression cassette and the second expression cassette are located within a single DNA molecule and are oriented in opposition directions; wherein different DNA strands serve as template strands during transcription for each expression cassette.

In some embodiments, the nucleotide sequence encoding the sense strand of the first expression cassette is flanked by the ITS and a reverse complement of the ITS and the antisense strand of the second expression cassette is flanked by the ITS and a reverse complement of the ITS. In some embodiments, the first expression cassette further comprises one or more terminator sequences downstream of the nucleotide sequence encoding the sense strand and the second expression cassette further comprises one or more terminator sequences downstream of the nucleotide sequence encoding the antisense strand. In some embodiments, the terminator sequence comprises a rrnBT1, rrnBT2, TT7, T7U, TT3, and/or PTH terminator sequence. In some embodiments, the terminator sequence comprises a nucleotide sequence of any one of SEQ ID NOs: 19-30.

In some embodiments, the construct further comprises a selection marker, optionally wherein the selection marker is located between the first expression cassette and the second expression cassette. In some embodiments, the selection marker is an antibiotic resistance selection marker or an antibiotic-free selection marker.

In some embodiments, either the promoter of the first expression cassette, the promoter of the second expression cassette, or both the promoter of the first expression cassettes and the promoter of the second expression cassettes is a bacteriophage T7 promoter.

In some embodiments, the construct is selected from plasmids, cosmids, bacterial artificial chromosomes, yeast artificial chromosomes, natural chromosomes, bacteriophages, and viruses. In some embodiments, the construct is a high, medium, or low copy number plasmid. In some embodiments, the plasmid is a PUC-based plasmid.

In some embodiments, the dsRNA targets a genomic sequence of an insect, a plant, a fungus, or a virus.

Some aspects of the present disclosure provide a construct comprising: (a) a first expression cassette comprising a promoter operably linked to an initial transcription sequence (ITS) comprising the nucleotide sequence of any one of SEQ ID NO: 1-4 or 38-41, a nucleotide sequence encoding a sense strand of a double-stranded RNA (dsRNA), and a terminator sequence; and (b) a second expression cassette comprising a promoter operably linked to an ITS comprising the nucleotide sequence of any one of SEQ ID NO: 1-4 or 38-41, a nucleotide sequence encoding an antisense strand of the dsRNA, and a terminator sequence, wherein the sense strand of the dsRNA is complementary to the antisense strand of the dsRNA.

In some embodiments, the first expression cassette and the second expression cassette are located within a single DNA molecule and are oriented in the same direction; and wherein the same DNA strand serves as the template strand during transcription for each expression cassette. In other embodiments, the first and second expression cassettes are located within a single DNA molecule; and wherein different DNA strands serve as template strands during transcription for each expression cassette.

In some embodiments, the nucleotide sequence encoding the sense strand of the first expression cassette is flanked by the ITS and a reverse complement of the ITS and the nucleotide sequence encoding the antisense strand of the second expression cassette is flanked by the ITS and a reverse complement of the ITS. In some embodiments, the first expression cassette further comprises one or more terminator sequences downstream of the nucleotide sequence encoding the sense and the second expression cassette further comprises one or more terminator sequences downstream of the nucleotide sequence encoding the antisense strand. In some embodiments, the terminator sequence comprises a rrnBT1, rrnBT2, TT7, T7U, TT3, and/or PTH terminator sequence. In some embodiments, the terminator sequence comprises a nucleotide sequence of any one of SEQ ID NOs: 19-30.

In some embodiments, the construct further comprises a restriction endonuclease recognition site downstream of the nucleotide sequence encoding the sense strand and/or the nucleotide sequence encoding the antisense strand, optionally downstream of either of the terminator sequences or in a construct lacking terminator sequences. In some embodiments, the construct further comprises a selection marker, optionally wherein the selection marker is located between the first expression cassette and the second expression cassette. In some embodiments, the selection marker is an antibiotic resistance selection marker or an antibiotic-free selection marker. In some embodiments, either the promoter of the first expression cassette, the promoter of the second expression cassette, or both the promoter of the first expression cassettes and the promoter of the second expression cassettes is a bacteriophage T7 promoter.

In some embodiments, the construct is selected from plasmids, cosmids, bacterial artificial chromosomes, yeast artificial chromosomes, natural chromosomes, bacteriophages, and viruses. In some embodiments, the construct is a high, medium, or low copy number plasmid. In some embodiments, the plasmid is a PUC-based plasmid.

Some aspects of the present disclosure provide an expression cassette comprising a promoter operably linked to a nucleotide sequence encoding a product of interest, wherein the nucleotide sequence is flanked by an initial transcription sequence (ITS) and optionally, two tandem terminator sequences and/or a restriction endonuclease site, wherein the ITS comprises the nucleotide sequence of any one of SEQ ID NOs: 1-8 or 38-41.

Some aspects of the present disclosure provide an engineered nucleic acid comprising the nucleotide sequence of any one of SEQ ID NO: 1-4 or 38-41.

Some aspects of the present disclosure provide an engineered nucleic acid comprising a promoter and an initial transcription sequence (ITS) comprising the nucleotide sequence of any one of SEQ ID NO: 1-4 or 38-41. In some embodiments, the engineered nucleic acid comprises the nucleotide sequence of any one of SEQ ID NO: 10-13 or 42-45.

Some aspects of the present disclosure provide a kit comprising: an engineered nucleic acid comprising the nucleotide sequence of any one of SEQ ID NO: 1-4 or 38-41; and a polymerase. In some embodiments, the kit further comprises nucleoside triphosphates and/or nucleoside monophosphates.

Some aspects of the present disclosure provide a vector or construct comprising a first expression cassette comprising a nucleotide sequence encoding the sense strand of a double stranded RNA (dsRNA) operably linked to a promoter, and a second expression cassette comprising a nucleotide sequence encoding the antisense strand of a dsRNA operably linked to a promoter, wherein the sense strand of the dsRNA is complementary to the antisense strand of the dsRNA.

Some aspects of the present disclosure provide vectors or constructs comprising a first expression cassette comprising a promoter operably linked to a first DNA initial transcription sequence (ITS) upstream of a nucleotide sequence encoding a sense strand of a double-stranded RNA (dsRNA), and a second expression cassette comprising a promoter operably linked to a second DNA ITS upstream of a nucleotide sequence encoding an antisense strand of the dsRNA, wherein the sense strand of the dsRNA is complementary to the antisense strand of the dsRNA.

Other aspects of the present disclosure provide vectors or constructs comprising a first expression cassette comprising a promoter operably linked to a first DNA initial transcription sequence (ITS) upstream of a nucleotide sequence encoding a sense strand of a double-stranded RNA (dsRNA) and a reverse complement of the DNA initial transcription sequence (ITS-RC) downstream of the nucleotide sequence encoding the sense strand of the dsRNA; and a second expression cassette comprising a promoter operably linked to a second DNA ITS upstream of a nucleotide sequence encoding an antisense strand of the dsRNA and a reverse complement of the DNA initial transcription sequence (ITS-RC) downstream of the nucleotide sequence encoding the antisense strand of the dsRNA. In some embodiments, the sense strand of the dsRNA is complementary to the antisense strand of the dsRNA and/or the ITS of each resultant RNA transcript corresponds to the DNA initial transcription sequence.

Other aspects provide vectors or constructs comprising a first expression cassette comprising a promoter operably linked to a first DNA initial transcription sequence (ITS) upstream of the nucleotide sequence encoding a sense strand of a double-stranded RNA (dsRNA), and at least one terminator sequence and/or a restriction endonuclease site; and a second expression cassette comprising a promoter operably linked to a second DNA initial transcription sequence (ITS) upstream of the nucleotide sequence encoding an antisense strand of the dsRNA, and at least one terminator sequence and/or a restriction endonuclease site. In some embodiments, the first and second expression cassettes are oriented in the same direction, the sense strand of the dsRNA is complementary to the antisense strand of the dsRNA, and the ITSes of each resultant RNA transcript corresponds to the DNA initial transcription sequence.

Other aspects provide vectors or constructs comprising a first expression cassette comprising a promoter operably linked to a first DNA initial transcription sequence (ITS) upstream of a nucleotide sequence encoding a sense strand of a double-stranded RNA (dsRNA), a reverse complement of the DNA initial transcription sequence (ITS-RC) downstream of the nucleotide sequence encoding the sense strand of a dsRNA, and at least one terminator sequence and/or restriction endonuclease site; and a second expression cassette comprising a promoter operably linked to a second DNA initial transcription sequence (ITS) upstream of a nucleotide sequence encoding an antisense strand of the dsRNA (dsRNA), a reverse complement of the DNA initial transcription sequence (ITS-RC) downstream of the nucleotide sequence encoding the antisense strand of a dsRNA, and at least one terminator sequence and/or restriction endonuclease site. In some embodiments, the sense strand of the dsRNA is complementary to the antisense strand of the dsRNA and/or the ITS of each RNA transcript corresponds to the DNA initial transcription sequence.

Some aspects of the present disclosure provide expression cassettes comprising a promoter operably linked to a DNA initial transcription sequence (ITS) upstream of a nucleotide sequence encoding a product of interest, and optionally, at least one terminator sequence and/or a restriction endonuclease site.

Other aspects of the present disclosure provide expression cassettes comprising a promoter operably linked to a DNA initial transcription sequence (ITS) upstream of a nucleotide sequence encoding a product of interest and a reverse complement of the DNA initial transcription sequence (ITS-RC) downstream of the nucleotide sequence encoding the product of interest.

In still other aspects, the present disclosure provides expression cassettes comprising a promoter operably linked to a DNA initial transcription sequence (ITS), upstream of a nucleotide sequence encoding a product of interest, a reverse complement of the DNA initial transcription sequence (ITS-RC) downstream of the nucleotide sequence encoding a product of interest, and at least one terminator sequence and/or a restriction endonuclease site. In some embodiments, the ITS comprises the nucleotide sequence of SEQ ID NO: 1.

Further aspects of the present disclosure provide nucleic acid architectural arrangement designs (e.g., a nucleic acid vector, nucleic acid construct). In some embodiments, a nucleic acid design comprises a plasmid, a linearized template, or any other DNA construct configuration, for the enhanced expression of a sequence of interest (e.g., an RNA of interest) (e.g., enhanced expression of a sequence of interest relative to a control construct). In some embodiments, the present disclosure provides an architectural design comprising a ‘complementary expression cassettes’ design (e.g., for expression of a dsRNA molecule of interest) involving two cassettes, wherein each cassette includes an ITS and optionally an ITS-RC, for the expression of a dsRNA molecule of interest. In some embodiments, an architectural design comprises a ‘complementary expression cassettes’ design involving two cassettes, wherein each cassette includes an ITS and optionally an ITS-RC, for the expression of a dsRNA molecule of interest, wherein the first expression cassette encodes the sense strand of the dsRNA, and the second expression cassette encodes the anti-sense strand, enabling the expression of both the sense and antisense strands, and wherein the first and second cassettes are encoded by two complementary strands of the same segment of double stranded DNA, wherein the sense and antisense RNA strands produced on transcription from the two cassettes anneal to generate a dsRNA molecule, which includes the r-ITS and optionally the r-ITC-RC. In some embodiments of this architecture, the sequence of interest, with or without a DNA ITS, is operably linked to two promoters on each end, with one promoter driving the expression of the sense strand of the desired dsRNA product, and the other driving the expression of the antisense strand of the desired dsRNA product. During transcription of the ‘complementary expression cassettes’ design, RNA polymerases begin transcription of the complementary strands from the promoters at the two ends, and the polymerases move toward each other while initially traversing the two complementary strands of DNA (e.g., in a converging manner).

In other embodiments, a nucleic acid architectural arrangement design (e.g., a vector or construct, e.g., for dsRNA expression) comprises an ‘independent expression cassettes’ design wherein the expression cassettes for the expression of sense and antisense strands of the dsRNA molecule are encoded by independent segments of DNA. The independent segments of DNA may be incorporated in the same plasmid, linearized template, or any other DNA construct. In some embodiments, an ‘independent expression cassettes’ design involves at least two expression cassettes that are part of the same vector or DNA molecule, wherein the first expression cassette and the second expression cassette are oriented in the same direction on the given vector or DNA molecule (the same DNA strand serves as a template strand during transcription from the respective promoters of both expression cassettes). In other embodiments, an ‘independent expression cassettes’ design involves at least two expression cassettes that are part of the same vector or DNA molecule, wherein the first expression cassette and the second expression cassette are oriented in opposite directions in the given vector or DNA molecule (e.g., the two opposite strands in the given vector or DNA molecule serve as template strands for the two expression cassettes). Depending on whether the two expression cassettes are oriented in the same direction or opposite directions on a given vector or other DNA molecule, the RNA polymerases may transcribe or function in the same direction on the same strand of the dsDNA molecule or in opposite directions on the two different strands of DNA.

In other embodiments, a nucleic acid architectural design (e.g., a vector or construct) comprises an ‘independent expression cassettes’ design involving two expression cassettes, wherein the expression cassettes for the expression of sense and antisense strands of a dsRNA molecule are encoded by independent segments of DNA, which may or may not be incorporated in the same plasmid, linearized template, or other DNA constructs. In some embodiments, an ‘independent expression cassettes’ design involves at least two expression cassettes that are part of the same vector or DNA molecule, including the ITS and optionally the ITS-RC, wherein the first expression cassette and the second expression cassette are oriented in the same direction on the given vector or DNA molecule (the same DNA strand serves as a template strand during transcription from the respective promoters of both expression cassettes). In some embodiments, an ‘independent expression cassettes’ design involves at least two expression cassettes that are part of the same vector or DNA molecule, including the ITS and optionally the ITS-RC, wherein the first expression cassette and the second expression cassette are oriented in opposite directions in the given vector or DNA molecule (e.g., the two opposite strands in the given vector or DNA molecule serve as template strands for the two expression cassettes). Depending on whether the two expression cassettes are oriented in the same direction or opposite directions on a given vector or DNA molecule, the RNA polymerases driving expression from the two independent promoters may transcribe or function in the same direction on the same strand of the dsDNA molecule or in opposite directions on the two different strands of DNA.

In other embodiments, a nucleic acid architectural arrangement design (e.g., a vector or construct) comprises a ‘multi-expression cassettes’ design, wherein multiple expression cassettes encoded by independent segments of DNA that are part of the same DNA molecule or different DNA molecules, allow for expression of multiple single-stranded RNA (ssRNA) molecules. In some embodiments, the multiple ssRNA molecules may encode the same sequence-of-interest (SOI) and/or may be incorporated in the same plasmid, linearized template, or any other DNA construct. In some embodiments, a ‘multi-expression cassettes’ design involves at least two expression cassettes that are part of the same vector or DNA molecule, wherein the first expression cassette and the second expression cassette are oriented in the same direction on the given vector or DNA molecule (the same DNA strand serves as a template strand during transcription from the respective promoters of both expression cassettes). In other embodiments, a ‘multi-expression cassettes’ design involves at least two expression cassettes that are part of the same vector or DNA molecule, wherein the first expression cassette and the second expression cassette are oriented in opposite directions (e.g., of the two strands in the vector or DNA molecule, a different or opposite DNA strand serves as a template strand during transcription from the respective promoters of both expression cassettes). In some embodiments, a ‘multi-expression cassettes’ design causes increased production of ssRNAs. Depending on whether the two expression cassettes are oriented in the same direction or opposite directions on a given vector or DNA molecule, the RNA polymerases driving expression from the two independent promoters may transcribe in the same direction on the same strand of the ssRNA molecule or in opposite directions on the two different strands of DNA.

In other embodiments, a nucleic acid arrangement architectural design (e.g., a vector or construct) comprises a ‘multi-expression cassettes’ design, wherein multiple expression cassettes are encoded by independent segments of DNA that are part of the same DNA molecule or different DNA molecules, and which each include an ITS and optionally, an ITS-RC, allowing for expression of multiple ssRNA molecules. The multiple ssRNA molecules may have the same SOI and/or may be incorporated into the same plasmid, linearized template, or any other DNA construct. In some embodiments, a ‘multi-expression cassettes’ design involves at least two expression cassettes that are part of the same vector or DNA molecule, wherein the first expression cassette and the second expression cassette are oriented in the same direction on the given vector or DNA molecule (the same DNA strand serves as a template strand during transcription from the respective promoters of both expression cassettes). In other embodiments, a ‘multi-expression cassettes’ design involves at least two expression cassettes that are part of the same vector or DNA molecule, wherein the first expression cassette and the second expression cassette are oriented in opposite directions (e.g., a different or opposite DNA strand serves as a template strand during transcription from the respective promoters of both expression cassettes). In some embodiments, a ‘multi-expression cassettes’ design leads to increased production of ssRNAs.

Also provided herein, in some aspects, are methods comprising combining in a transcription reaction, any one of the vectors or constructs described herein and a polymerase, and producing an RNA transcript.

### BRIEF DESCRIPTION OF THE DRAWINGS

FIG. 1 provides a schematic of an exemplary plasmid DNA template with an expression cassette for in vitro or in vivo transcription of RNA products.

FIG. 2 provides schematics of exemplary linear DNA templates for in vitro or in vivo transcription of RNA products, wherein the DNA templates each comprise expression cassettes comprising a promoter operably linked to an ITS upstream of a sequence-of-interest (SOI) encoding the sense or antisense strand of a dsRNA product. The resultant dsRNA product, when completely hybridized, comprises 5′ r-ITS overhangs that correspond to each ITS of the DNA template.

FIG. 3 provides schematics of exemplary linear DNA templates for in vitro or in vivo transcription of RNA products containing a 5′ r-ITS and a 3′ r-ITS-RC in the resulting RNA transcripts. The two DNA templates shown each comprise expression cassettes encoding the sense and antisense strands respectively of a dsRNA product, wherein each expression cassette comprises a promoter operably linked to an ITS upstream of a sequence-of-interest (SOI) encoding either the sense or antisense strands and an ITS-reverse complement (ITS-RC). The resultant dsRNA product, when completely hybridized, does not comprise any single-stranded overhangs.

FIG. 4 provides schematics of exemplary DNA templates for in vitro or in vivo transcription to produce dsRNA products. The top schematic is a DNA template comprising the ‘complementary expression cassettes’ architecture, wherein the expression cassettes for the sense and antisense strands of the dsRNA product are encoded by the two complementary DNA strands and wherein the DNA template comprises two converging promoters operably linked to ITS_2 sequences positioned on the two ends of a sequence-of-interest (SOI) to be transcribed. The middle schematic is a DNA template comprising the ‘complementary expression cassettes’ architecture for dsRNA synthesis, wherein the expression cassettes for the sense and antisense strands of a dsRNA product are encoded by two complementary DNA strands and wherein the DNA template comprises two converging promoters with GL-Hybrid-A9 ITSes positioned on the two ends of a sequence-of-interest (SOI) to be transcribed. The bottom schematic is a DNA template comprising the ‘independent expression cassettes’ architecture for dsRNA synthesis, wherein the sense and antisense strands of the dsRNA product are encoded by separate expression cassettes on separate segments of DNA, wherein each expression cassette comprises of a promoter and ITS positioned upstream of a sequence-of-interest (SOI) encoding either the sense or antisense strand to be transcribed.

FIG. 5 is a graph showing expression levels (titers, in ng/μL) of a dsRNA product (GS1 dsRNA) obtained following in vitro transcription (IVT) reactions using DNA templates comprising differing ITSes.

FIGS. 6A-6B are graphs showing the expression levels (titers, in ng/μL) of a dsRNA product (GS1 dsRNA) obtained following cell-free reactions using DNA templates comprising differing ITSes. Data was obtained using DNA templates that generate dsRNA products with 5′ single stranded overhangs (FIG. 6A) and without 5′ single stranded overhangs (FIG. 6B).

FIGS. 7A-7D provide graphs showing fold increases in expression levels of dsRNA products (GL Seq-A, GL Seq-B, GL Seq-C, GL Seq-D, GL Seq-E) using various DNA template architectures. FIG. 7A is a graph showing RNA titer proxies using different DNA template architectures. FIG. 7B shows fold increases in expression levels of dsRNA for DNA templates using the ‘complementary expression cassettes’ design with a GL-Hybrid_A9 ITS relative to DNA templates employing the same design without a GL-Hybrid_A9 ITS. FIG. 7C shows fold increases in expression levels for DNA templates using the ‘independent expression cassettes’ design with a GL-Hybrid_A9 ITS relative to DNA templates using the ‘complementary expression cassettes’ design with same GL-Hybrid_A9 ITS. FIG. 7D shows fold increases in expression levels for DNA templates using the ‘independent expression cassettes’ design with a GL-Hybrid_A9 ITS relative to DNA templates using the ‘complementary expression cassettes’ design without a GL-Hybrid_A9 ITS.

FIGS. 8A-8C demonstrate the effectiveness of differing terminator sequences to terminate transcription of single-stranded RNA (ssRNA) products. FIG. 8A provides schematics of DNA templates used for evaluation of read-through and termination efficiency. FIG. 8B provides reverse-phase ion-pair (RP-IP) High-performance liquid chromatography (HPLC) chromatograms of ssRNA products synthesized in in vitro transcription reactions using the DNA templates shown in FIG. 8A. FIG. 8C is a graph showing the net termination efficiency of in vitro transcription reactions using the DNA templates shown in FIG. 8A at varying levels of nucleoside triphosphates (NTPs) (2 mM, 4 mM, and 8 mM of each NTP).

FIG. 9 is a schematic of an exemplary DNA plasmid employing the ‘independent expression cassettes’ design for transcription of a sense and antisense strand of a dsRNA product from two separate expression cassettes. Transcription of the sense and antisense strands are independently driven from a T7 promoter operably linked to a DNA initial transcribed sequence (ITS), as described herein.

FIG. 10 is a graph showing the expression levels (titers, in ng/μL) of a dsRNA product (GS4 dsRNA) obtained following cell-free reactions using plasmid (pGLA583 & pGLA584) and linear DNA templates.

FIG. 11 is a graph showing the expression levels (titers, in ng/μL) of a dsRNA product (GS1 dsRNA) obtained following cell-free reactions (NTP reactions and NMP reactions) using DNA templates with various ITSes.

FIG. 12 is a graph showing the expression of a dsRNA product (GLSeq-A dsRNA) from a plasmid DNA template comprising two independent expression cassettes encoding the sense and the antisense strands (Plasmid Construct-1) compared to expression of the GLSeq-A dsRNA product as a “hairpin” product variant from a plasmid DNA template comprising a single expression cassette (Plasmid Construct-2).

FIGS. 13A-13B are schematics of exemplary plasmid DNA templates for the expression of dsRNA products. FIG. 13A shows an exemplary plasmid DNA template (Plasmid Construct-3) employing the ‘independent expression cassettes’ architecture for dsRNA production, wherein each of the two separate expression cassettes in the plasmid respectively allow expression of the sense and antisense strands of a dsRNA product flanked by an ITS and ITS-RC. The two expression cassettes are oriented in the same direction and separated by the ampicillin resistance bla gene as a selection marker and an origin of replication. FIG. 13B shows an exemplary plasmid DNA template (Plasmid Construct-4) employing the “complementary expression cassettes” architecture for dsRNA expression, wherein two complementary DNA strands of the same segment of DNA allow expression of the sense and antisense strands of the dsRNA product and the segment of DNA encoding the two expression cassettes comprises the sequence of interest (SOI) flanked on each end by T7 promoters operably linked to a suitable ITS.

FIG. 14 is a graph showing the expression levels (titers, in ng/μL) of two different dsRNA products (GS1 and GS4) obtained from cell-free reactions using plasmid DNA templates employing either the ‘independent expression cassettes’ architecture (Plasmid Construct-3) or the “complementary expression cassettes” architecture (Plasmid Construct-4)

FIG. 15 is a graph showing the expression levels (titers, in ng/μL) of GS1 dsRNA obtained from cell-free reactions using plasmid DNA templates employing the ‘independent expression cassettes’ architecture (Plasmid Construct-3) with different ITS variants used as part of the expression cassettes.

FIG. 16A is a graph showing the production titers (titers, in mg/ml) of uncapped RNA produced using a G-start ITS and capped RNA with an A-start ITS. FIG. 16B shows electropherograms illustrating the size distribution of RNA species produced in the cell-free reactions, captured using a BioAnalyzer instrument.

FIG. 17A is a graph showing the production titers (titers, in mg/ml) of capped RNA using CleanCap AG, A-start ITS, and various open reading frame sequences. FIG. 17B shows electropherograms illustrating the size distribution of RNA species produced in the cell-free reactions, captured using a Fragment Analyzer instrument.

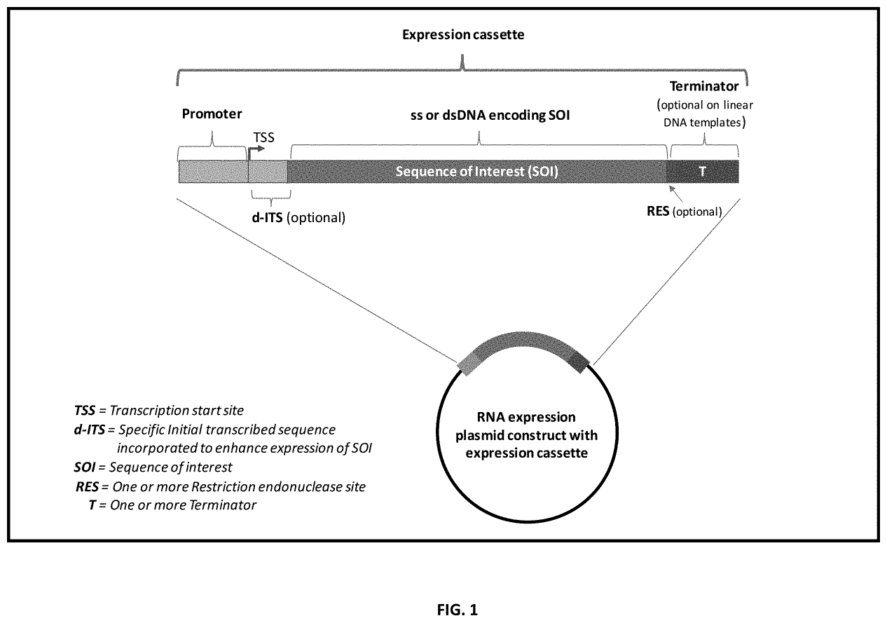

### DETAILED DESCRIPTION

Provided herein, in some aspects, are nucleic acids, compositions of nucleic acids, e.g., DNA-based vectors or constructs, and associated methods of use and kits for the production of ribonucleic acid (RNA), such as double-stranded RNA (dsRNA) and single-stranded RNA (ssRNA). The compositions provided herein enable cost-effective and high-yield production of RNA using cell-free reactions and in vitro transcription reactions.

### Definitions

While the following terms are believed to be well understood by one of ordinary skill in the art, the following definitions are set forth to facilitate explanation of the presently disclosed subject matter.

The terms “nucleic acid” or “nucleic acid molecule,” as used herein, generally refers to deoxyribonucleic acid (DNA) or ribonucleic acid (RNA). A nucleic acid may be single-stranded or double-stranded. The nucleotide monomers in the nucleic acid molecules may be naturally occurring nucleotides, modified nucleotides or combinations thereof. Modified nucleotides, in some embodiments, comprise modifications of the sugar moiety and/or the pyrimidine or purine base.

The term “transcription” or “RNA transcription” generally refers to the process by which RNA transcripts are synthesized by an RNA polymerase that is capable of polymerizing ribonucleoside triphosphates using a nucleic acid molecule (DNA or RNA) as a template, either in vivo or in vitro.

The terms “template” or “transcription template” or “template for transcription” generally refers to a nucleic acid sequence (DNA or RNA) that serves as a template for an RNA polymerase to make RNA transcripts via the process of transcription. The template specifies the sequence of the RNA transcripts that are synthesized by the RNA polymerase. The RNA polymerase synthesizes RNA transcripts by moving along the template strand of the template nucleic acid molecule and adding ribonucleotide triphosphates complementary to the template (DNA or RNA) strand, to a growing RNA transcript. The template may be DNA or RNA. In some embodiments, the template is single-stranded or double-stranded. In most living organisms, transcription is carried out by RNA polymerases using double stranded DNA molecules (chromosomal DNA) as the template in cells to synthesize mRNA. In some embodiments, in vitro transcription utilizes synthetic partially double stranded DNA templates to be transcribed by DNA-dependent RNA polymerases. In some embodiments, a template is a linear molecule. In some embodiments, a template is circular. A template may contain additional elements other than those necessary for expression of RNA transcripts. Additionally, in vivo and/or in vitro transcription from single stranded RNA by RNA dependent RNA polymerases is also possible (e.g. as in case of some RNA viruses). The terms “template” or “transcription template” or “template for transcription” may, in some embodiments, refer either to a specific nucleic acid sequence of a segment of a double-stranded DNA molecule or to an entire DNA molecule that contains a nucleic acid sequence to be transcribed.

The terms “T7 promoter”, “T7 RNAP promoter” or “T7 Class III promoter” generally refer to a double stranded DNA segment that a T7 RNA polymerase binds to in order to initiate transcription. In some embodiments, a T7 promoter is a minimal T7 class III promoter. In some embodiments, a minimal T7 class III promoter is a 17 base pair (bp) long dsDNA segment naturally found upstream of the φ6.5, φ10 and φ13 genes in the T7 bacteriophage genome, the non-template strand of which has the sequence TAATACGACTCACTATA (SEQ ID NO: 9). The minimal T7 class III promoter as defined herein does not include the canonical ‘G’ at its end and is not expected to initiate transcription on its own, unless the sequence to be expressed carries a ‘G’ at its beginning. In some embodiments, a T7 promoter comprises a 47 bp promoter comprising: a) 30 bp DNA segment (denoted by the non-template strand sequence TCGATTCGAACTTCTGATAGACTTCGAAAT; SEQ ID NO: 37) that is naturally found upstream of the minimal T7 class III promoter in the regions preceding the φ6.5, φ10 and φ13 genes in the T7 bacteriophage genome operably linked to b) 17 bp minimal T7 class III promoter of SEQ ID NO: 9.

The term “transcription Start Site (TSS)” generally refers to the specific nucleotide location on the template where the RNA polymerase initiates transcription. The transcription start site is generally the nucleotide location immediately downstream of the promoter, at which synthesis of RNA by the RNA polymerase starts. In some embodiments, in instances in which the template for transcription is a DNA molecule with a promoter operably linked to an ITS, the TSS is the first nucleotide of the ITS. In some embodiments, in instances in which the template for the transcription is a DNA molecule with a promoter operably linked to a sequence of interest (i.e., template lacking an ITS), the TSS is the first nucleotide of the sequence of interest. As used herein, the TSS generally does not overlap with the minimal T7 class III promoter but is included as the first nucleotide of the ITS or, if an ITS is not present, the first nucleotide of the sequence of interest.

The term “sequence of interest (SOI)” generally refers to a specific nucleic acid sequence that is incorporated into an RNA transcript or RNA product produced via transcription. Thus, in some embodiments, a SOI is a segment of the DNA template that encodes a specific nucleic acid sequence of an RNA product. In some embodiments, a SOI is nucleic acid sequence of a part or whole of an RNA transcript or product.

The term “DNA initial transcription sequence (ITS)” generally refers to a sequence comprising the first several nucleotides (e.g., 1-15 nucleotides) of the transcribed sequence on the DNA template, immediately downstream of the promoter. In some embodiments, the DNA ITS influences the overall yield of full-length RNA transcripts produced via transcription (e.g., increases the overall yield relative to a control DNA template lacking an ITS sequence). After initial binding to the promoter, an RNA polymerase is expected to cycle back and forth over the ITS to release short abortive RNA transcripts, before promoter clearance and transition to the elongation phase of transcription, allowing for synthesis of full-length RNA transcripts.

The term “RNA initial transcript sequence (r-ITS)” generally refers to the sequence comprising the first several nucleotides (e.g., 1-15 nucleotides) at the beginning of an RNA transcript, corresponding to the ITS on the DNA template used for transcription of said RNA transcript. The r-ITS may refer to a sequence present at the beginning of RNA transcripts upstream of a SOI. In some embodiments, the r-ITS corresponds to a naturally-occurring ITS. In some embodiments, the r-ITS is a heterologous or synthetic sequence that is present between a promoter and SOL

The term “ITS” generally refers to a DNA initial transcription sequence (ITS). The term “ITS-RC” generally refers to the reverse complement of a DNA initial transcription sequence. The term “r-ITS” generally refers to an RNA initial transcription sequence that has been transcribed from a corresponding ITS. The term “r-ITS-RC” generally refers to the reverse complement of an RNA initial transcription sequence that has been transcribed from a corresponding ITS-RC.

As used herein, the terms “transcriptional terminator” or “terminator” generally refer to a specific sequence, typically at the end of a SOI on a DNA template that causes RNA transcription by a polymerase to terminate. In some embodiments, the terminator is a nucleic acid sequence that causes an RNA polymerase to release from a DNA template and transcription to stop. A terminator may be unidirectional or bidirectional.

The terms “sense” and “antisense” generally refer to the individual strands in double stranded DNA or RNA molecules. The term “sense strand” as used herein generally refers to the nucleic acid sequence of the coding strand of a double-stranded nucleic acid molecule. The term “antisense strand” may be used to refer to the nucleic acid sequence of the template strand, or a segment thereof, of a double-stranded nucleic acid that is transcribed to produce mRNA. Alternatively, the term “antisense strand” may refer to the nucleic acid sequence of an RNA strand that is complementary to an mRNA transcript or a segment thereof.

The term “expression cassette” generally refers to a DNA sequence that serves as a DNA template for expression of an RNA transcript of interest via transcription. In some embodiments, an expression cassette is at least composed of a promoter operably linked to a nucleic acid sequence encoding the RNA molecule to be expressed. The expression cassette optionally also includes one or more of the following elements: a specific initial transcription sequence (ITS) to enhance expression, a reverse complement of the specific ITS (ITS-RC), one or more restriction endonuclease site(s) (RES), and/or one or more terminator(s).

As used herein, the terms “construct”, “nucleic acid construct”, “expression construct”, “engineered nucleic acid” or “vector” generally refer to a DNA molecule which includes one or more expression cassettes for the expression of an RNA transcript or product of interest (e.g., an RNA product or a protein-of-interest) via transcription by an RNA polymerase, in vitro or in vivo. “Construct” and “vector” are used interchangeably herein. A construct may include additional elements that are not critical for expression of the RNA transcript, but essential for ensuring its own replication, maintenance, stability etc. in vivo or in vitro. For example, a construct may be a plasmid with one or more expression cassettes that additionally has an origin of replication and a selection marker for its replication and maintenance in a suitable host, respectively. Alternatively, the chromosome of an organism that has been modified by integrating one or more expression cassettes, to allow expression of the RNA transcripts may comprise a construct. Non-limiting examples of constructs thus include, vectors, viral vectors (e.g., adeno-associated viral vectors), plasmids, cosmids, plastomes, bacteriophages, artificial chromosomes, natural genomes with expression cassettes integrated, or linear DNA molecules.

As used herein, in some embodiments, two nucleic acid sequences or elements are considered to be “operably linked” when the two nucleic acid sequences or elements are functionally connected to one another. For example, in some embodiments, a promoter is operably linked to an initial transcription sequence (ITS) such that the promoter is in a correct functional location and orientation in relation to the ITS that it regulates to control (“drive”) transcriptional initiation and/or expression of that ITS sequence.

### Production of RNA

Transcription of, e.g., production of, RNA involves three main steps. During the first step (initiation), an RNA polymerase binds to the promoter on a DNA template, melts the two strands of DNA (a non-template strand and a template strand) and initiates transcription at the transcription start site (TSS) by incorporating and polymerizing ribonucleotides complementary to the template DNA strand. The polymerase may continue to cycle back and forth over the first few nucleotides to release short abortive transcripts (3 to 8 nucleotides in length) repeatedly until successfully transitioning to the next step (elongation). Following promoter clearance and formation of a stable ternary elongation complex, the polymerase continues to move along the DNA template and elongate/build the RNA product by incorporating ribonucleotides complementary to the template DNA strand. Following complete production of the RNA product, the third step (termination) involves release of the polymerase from the transcribed RNA product as a result of the polymerase encountering a terminator sequence on the DNA template or by reaching the end of a linear DNA template and falling off the template.

Described herein are compositions of nucleic acids, e.g., DNA templates, for use in methods of RNA transcription. In some embodiments, a nucleic acid, e.g., a DNA template, comprises template elements, e.g., a DNA initial transcribed sequence (ITS), that improve the efficiency of each of the steps of transcription, e.g., initiation, elongation, and/or termination, to maximize RNA product yield from transcription reactions, e.g., cell-free reactions and/or in vitro or in vivo transcription reactions. In some embodiments, a nucleic acid, e.g., a DNA template, comprises an ITS of variable length and nucleotide sequences. In some embodiments, a nucleic acid, e.g., a DNA template, e.g., a DNA vector, DNA construct or DNA plasmid, comprises two expression cassettes, wherein one expression cassette encodes a sense strand of a double-stranded RNA (dsRNA) and the other expression cassette encodes an antisense strand of said dsRNA. In some embodiments, the sense strand of the dsRNA is completely or partially complementary to the antisense strand of the dsRNA. In some embodiments, a nucleic acid, e.g., a DNA template, e.g., a DNA vector, DNA construct or DNA plasmid, comprises at least two expression cassettes, wherein each expression cassette encodes a SOI to produce ssRNA molecules.

In some embodiments, a promoter region of a DNA template is operably linked to a DNA initial transcribed sequence (ITS). The ITS is a short sequence up to 15 nucleotides (e.g., 6-15 nucleotides) that influences the transition from the initiation phase of transcription to the elongation phase via promoter clearance, thereby influencing the rate and net yield of transcription from a given promoter. In some embodiments, the ITS is initially and repeatedly transcribed to release short abortive transcripts during the initiation step of transcription. An r-ITS is consequently present at the 5′ end of a full-length RNA transcript following transcription of the ITS on the DNA template. Thus, an ITS, when present, has a critical role in the early stages of transcription (initiation and the transition to elongation phase via promoter clearance) and influences the overall rate and yield of transcription from a given promoter. In some embodiments, an ITS is a naturally occurring ITS, e.g., a consensus ITS found immediately after a T7 class III promoter in the bacteriophage T7 genome. In some embodiments, a consensus ITS precedes the φ6.5, φ10 and φ13 genes and comprises the first 6 nucleotides (GGGAGA (SEQ ID NO: 8)) immediately downstream of a T7 class III promoter. In some embodiments, an ITS is a synthetic ITS (e.g., GGGAGACCAGGAATT (SEQ ID NO: 1).

A promoter may be naturally associated with a gene or sequence, e.g., an endogenous promoter. In some embodiments, an endogenous promoter is located upstream of the coding segment of a given gene or sequence. In some embodiments, a coding nucleic acid sequence, e.g., an SOI, may be positioned under the control of a recombinant or heterologous promoter, which refers to a promoter that is not normally associated with the encoded sequence in its natural environment. Such promoters may include promoters of other genes; promoters isolated from any other species; and synthetic promoters or enhancers that are not “naturally occurring” such as, for example, those that contain different elements of different transcriptional regulatory regions and/or mutations that alter expression through methods of genetic engineering that are known in the art.

In some embodiments, RNA is produced using the nucleic acids described herein and a cell-free reaction, such as is described in International Publication No. WO 2019/075167. In some embodiments, a cell-free transcription reaction involves three main processes: (1) the degradation of intracellular polymeric RNA into nucleotide monomers—a combination of nucleotide monophosphates (NMPs) and nucleotide diphosphates (NDPs), (2) the conversion of the NMPs and NDPs to nucleotide triphosphates (NTPs), which serve as “building blocks” for the formation of polymeric RNA, and (3) the polymerization of the NTPs to produce RNA using a composition of nucleic acids, e.g., a DNA-based vector.

In some embodiments, RNA is produced using the composition of a nucleic acid described herein and an in vitro transcription (IVT) reaction. In some embodiments, an IVT reaction comprises a recombinant RNA polymerase, NTPs, salts, metals, cofactors, and/or buffers. In some embodiments, any described IVT reaction in the art may be used with the compositions of nucleic acids described herein.

Methods of producing RNA may be performed at a temperature of 4° C. to 80° C., or higher. For example, methods of producing RNA may be performed at a temperature of 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C. or 80° C. Methods of producing RNA may be performed for a period of time of 5 minutes (min) to 48 hours (hr), or more. For example, methods of producing RNA may be performed for a period of time of 5 min, 10 min, 15 min, 20 min, 30 min, 45 min, 1 hr, 2 hrs, 3 hrs, 4 hrs, 5 hrs, 6 hrs, 7 hrs, 8 hrs, 9 hrs, 10 hrs, 11 hrs, 12 hrs, 18 hrs, 24 hrs, 30 hrs, 36 hrs, 42 hours, or 48 hours.

Methods of producing RNA may be catalyzed, in some embodiments, by a highly processive DNA-dependent T7 RNA polymerase, e.g., as encoded from gene 1 from the T7 bacteriophage genome. In some embodiments, an RNA polymerase is a T7 RNA polymerase from T7 phage. In some embodiments, an RNA polymerase is an RNA-dependent RNA polymerase, e.g., Φ6 RNA polymerase from phage Φ6. Other DNA-dependent or RNA-dependent RNA polymerases may be used in accordance with the present disclosure.

In some embodiments, transcription of a nucleic acid of the current disclosure produces an RNA product, e.g., a sense strand or antisense strand of a dsRNA, a messenger RNA, shRNA, siRNA, ssRNA, gRNA, an antisense oligonucleotide, or a gapmer. In some embodiments, transcription of a nucleic acid of the current disclosure produces an RNA product that is flanked by an ITS (r-ITS) and a reverse complement of the ITS (r-ITS-RC).

In some embodiments, methods of producing RNA using the compositions described herein produces at least 5% more RNA, at least 10% more RNA, at least 20% more RNA, at least 30% more RNA, at least 40% more RNA, at least 50% more RNA, at least 60% more RNA, at least 70% more RNA, or greater, than a control. For example, methods using a vector or construct comprising a promoter operably linked to an ITS, e.g., an ITS comprising the nucleotide sequence of SEQ ID NO: 1, produces at least 5% more RNA, at least 10% more RNA, at least 20% more RNA, at least 30% more RNA, at least 40% more RNA, at least 50% more RNA, at least 60% more RNA, at least 70% more RNA or greater, than a control (e.g., methods using a vector or construct that does not comprise a promoter operably linked to an ITS).

In some embodiments, RNA is produced via transcription using the compositions of nucleic acid constructs or engineered nucleic acids described herein, e.g., in a microbial cell comprising a nucleic acid construct or engineered nucleic acid described herein. In some embodiments, the nucleic acid construct or engineered nucleic acid is integrated into the chromosome of the microbial cell. In some embodiments, the nucleic acid construct or engineered nucleic acid is a plasmid or any other nucleic acid construct or engineered nucleic acid contained within the microbial cell. In some embodiments, RNA is produced in a prokaryotic or eukaryotic cell comprising a nucleic acid construct described herein, grown under conditions optimal for RNA production. In some embodiments, the cells comprising the nucleic acid construct or engineered nucleic acid described herein are grown in fermentation chambers or reactors to produce RNA as a product of interest. In other embodiments, the cells comprising the nucleic acid construct or engineered nucleic acid described herein are grown in fermentation chambers or reactors for the production of protein or peptides of interest, wherein the nucleic acid construct or engineered nucleic acid allow expression of RNA encoding the protein or peptide product of interest inside the cells.

### Initial Transcribed Sequence

An ITS for use in compositions of nucleic acids described herein may comprise 1-15 nucleotides in length, e.g., 1, 2, 3, 4, 5, 6, 7, 8, 9, 10, 11, 12, 13, 14, or 15 nucleotides in length. In some embodiments, an ITS is 1-6, 1-9, 1-12, 1-15, 3-6, 6-9, 6-12, 6-15, 9-15, 9-12, 10-15, 10-12, 12-15, or more than 15 nucleotides in length. In some embodiments, an ITS is located immediately downstream of a promoter, e.g., a T7 class III minimal promoter. In some embodiments, an ITS encompasses a transcription start site.

In some embodiments, an ITS is as described in Table 1. In some embodiments, an ITS is any one of SEQ ID NO: 1-8 or 38-41. In some embodiments, an ITS is any one of SEQ ID NO: 1-4 or 38-41. In some embodiments, an ITS is a variant of any one of SEQ ID NO: 1-8 or 38-41, wherein the variant comprises at least 1, 2, 3, 4, 5, or more mutations. In some embodiments, a promoter operably linked to an ITS is located upstream of a sequence-of-interest (SOI), wherein the SOI encodes for a desired RNA product, e.g., a sense or antisense strand of a dsRNA. In some embodiments, a T7 class III minimal promoter comprises a TAATACGACTCACTATA (SEQ ID NO: 9). In some embodiments, a T7 class III minimal promoter is preceded by a 30 base pair sequence naturally present upstream of the promoter in the T7 bacteriophage genome region, e.g., comprising TCGATTCGAACTTCTGATAGACTTCGAAATTAATACGACTCACTATA (SEQ ID NO: 18).

TABLE 1
ITS variants

| | ITS (Initial | |
|---|---|---|
| | Transcribed | Minimal T7 promoter |
| Name | Sequence) | operably linked to ITS |
| ITS_6 | GGGAGA | TAATACGACTCACTATAGGGAGA (SEQ |
| | (SEQ ID NO: 8) | ID NO: 17) |
| pT7-g10 | GGGAGACCACA | TAATACGACTCACTATAGGGAGACCAC |
| | ACGT (SEQ ID NO: | AACGT (SEQ ID NO: 14) |
| | 5) | |
| pT7-g5 | GGGAGACCGGA | TAATACGACTCACTATAGGGAGACCGG |
| | ATT (SEQ ID NO: | AATT (SEQ ID NO: 15) |
| | 6) | |
| pT7- | GGGAGACCGGA | TAATACGACTCACTATAGGGAGACCGG |
| 5_LIT_AI | ATTT (SEQ ID NO: | AATTT (SEQ ID NO: 16) |
| | 7) | |
| GL- | GGGAGACCAGG | TAATACGACTCACTATAGGGAGACCAG |
| hybrid_A9 | AATT (SEQ ID NO: | GAATT (SEQ ID NO: 10) |
| | 1) | |
| GL- | GGGAGACCGGG | TAATACGACTCACTATAGGGAGACCGG |
| hybrid_A9G | AATT (SEQ ID NO: | GAATT (SEQ ID NO: 11) |
| | 2) | |
| GL- | GGGAGACCCGG | TAATACGACTCACTATAGGGAGACCGG |
| hybrid_A9C | AATT (SEQ ID NO: | GAATT (SEQ ID NO: 12) |
| | 3) | |
| GL- | GGGAGACCTGG | TAATACGACTCACTATAGGGAGACCTGG |
| hybrid_A9T | AATT | AATT (SEQ ID NO: 13) |
| | (SEQ ID NO: 4) | |
| GL- | AGGAGACCAGG | TAATACGACTCACTATAAGGAGACCAG |
| hybrid_A9 | AATT (SEQ ID NO: | GAATT (SEQ ID NO: 42) |
| (A start) | 38) | |
| GL- | AGGAGACCGGG | TAATACGACTCACTATAAGGAGACCGG |
| hybrid_A9G | AATT (SEQ ID NO: | GAATT (SEQ ID NO: 43) |
| (A start) | 39) | |
| GL- | AGGAGACCCGG | TAATACGACTCACTATAAGGAGACCCGG |
| hybrid_A9C | AATT (SEQ ID NO: | AATT (SEQ ID NO: 44) |
| (A start) | 40) | |
| GL- | AGGAGACCTGGA | TAATACGACTCACTATAAGGAGACCTGG |
| hybrid_A9T | ATT | AATT (SEQ ID NO: 45) |
| (A start) | (SEQ ID NO: 41) | |

In some embodiments, the promoter is a Minimal Class III T7 Promotor comprising a sequence: TAATACGACTCACTATA (SEQ ID NO: 9). In some embodiments, the promoter is an Extended Class III T7 Promoter comprising a sequence:

(SEQ ID NO: 18)
TCGATTCGAACTTCTGATAGACTTCGAAATTAATACGACTCACTATA.

The ITS_6 variant corresponds to a conserved region of the ITS preceding the genes φ6.5, φ10 and φ13 genes from the T7 genome. The pT7-g10, pT7-g5, and pT7-5_LIT_AI variants correspond to previously reported synthetic or naturally occurring ITSes. The GL-hybrid variants (GL-hybrid_A9, GL-hybrid_A9G, GL-hybrid_A9C, GL-hybrid_A9T) were identified by the inventors of the current disclosure and are surprisingly effective in promoting RNA transcriptional processes. As described by the Examples, DNA templates comprising a promoter operably linked to a GL-hybrid ITS variant produced higher levels of transcribed RNA products relative to control DNA templates, e.g., DNA template comprising ITS_6.

### Sequence of Interest

The compositions of nucleic acids, e.g., DNA templates, described herein comprise a sequence-of-interest (SOI), wherein the SOI is any sequence that encodes an RNA product. In some embodiments, an SOI is operably linked to a promoter, optionally a promoter comprising an ITS. The promoter drives expression or drives transcription of the SOI that it regulates.

In some embodiments, an RNA product is a sense strand of a double-stranded RNA (dsRNA). In some embodiments, an RNA product is an antisense strand of a dsRNA. In some embodiments, a sense strand of a dsRNA is complementary to an antisense strand of a dsRNA. In some embodiments, an RNA product is a single-stranded RNA, e.g., messenger RNA. In some embodiments, an RNA product is shRNA, siRNA, an antisense oligonucleotide, a gapmer, or any other conceivable RNA product.

In some embodiments, an RNA product, e.g., a dsRNA, targets (e.g. via RNA interference) a genomic sequence of interest, e.g., from an insect, a plant, a fungus, an animal, or a virus. In some embodiments, an RNA product, e.g., an mRNA, encodes a protein of interest.

In some embodiments, an SOI that encodes an RNA product may have any length sufficient to induce biological activity. Non limiting examples may include an SOI that encodes an RNA product with a length of 4 to 10, 4 to 20, 4 to 30, 4 to 50, 4 to 60, 4 to 70, 4 to 80, 4 to 90, 4 to 100, 4 to 200, 4 to 300, 4 to 400, 4 to 500, 4 to 1000, 500 to 2000 nucleotides, 500 to 4000 nucleotides, 500 to 6000 nucleotides, 500 to 8000 nucleotides, or 4 to 10000 nucleotides. In some embodiments, an SOI that encodes an RNA product has a length of 15, 16, 17, 18, 19, 20, 21, 22, 23, 24, or 25 nucleotides. In some embodiments, an SOI that encodes an RNA product has a length of 4, 5, 6, 7, 8, 9, 10, 15, 20, 25, 30, 35, 40, 45, 50, 55, 60, 65, 70, 75, 80, 85, 90, 95, 100, 500, 1000, or more nucleotides.

Two nucleic acids, e.g., the sense and antisense strands of dsRNA, are complementary (e.g., wholly or partially) to one another if they base-pair, or bind, to each other to form a double-stranded nucleic acid molecule via Watson-Crick interactions (also referred to as hybridization). As used herein, binding refers to an association between at least two molecules or two regions of the same molecule due to, for example, electrostatic, hydrophobic, ionic, and/or hydrogen-bond interactions under physiological conditions. In some embodiments, the two nucleic acids are 100% complementary (i.e., wholly complementary along a segment or the entirety of the nucleic acids). In some embodiments, the two nucleic acids are at least 75%, 80%, 85%, 90%, or 95% complementary (e.g., partially complementary) along a segment or the entirety of the nucleic acids. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies), ELAND (Illumina, San Diego, Calif.), SOAP (available at soap.genomics.org.cn), and Maq (available at maq.sourceforge.net). In some embodiments, two nucleic acids that are complementary to one another comprise 1, 2, 3, 4, 5, 6, 7, 8, 9, 10, or more mismatched base pairs.

In some embodiments, a double-stranded RNA or dsRNA is a wholly double-stranded molecule, which does not contain a single-stranded region (e.g., a loop or overhang). In some embodiments, a double-stranded RNA or dsRNA is a partially double-stranded molecule, which contains a double-stranded region and a single-stranded region (e.g., a loop or overhang).

### Terminator Sequence

In some embodiments, a composition of a nucleic acid, e.g., a DNA template, includes a transcriptional terminator sequence. The sequence encoding for the transcriptional terminator is typically located immediately downstream of the coding sequence. It is comprised of a DNA sequence involved in specific termination of an RNA transcript by an RNA polymerase. A terminator sequence prevents transcriptional activation of downstream nucleic acid sequences by upstream promoters. Thus, in some embodiments, DNA templates comprising a terminator that ends the production of an RNA transcript are contemplated. The most commonly used type of terminator is a forward terminator. When placed downstream of a nucleic acid sequence that is usually transcribed, a forward transcriptional terminator will cause transcription to abort. In some embodiments, bidirectional transcriptional terminators are provided, which usually cause transcription to terminate on both the forward and reverse strand. In some embodiments, reverse transcriptional terminators are provided, which usually terminate transcription on the reverse strand only. In prokaryotic systems, terminators usually fall into two categories: (1) rho-independent terminators, and (2) rho-dependent terminators. Rho-independent terminators are generally composed of palindromic sequence that forms a stem loop rich in G-C base pairs followed by a string of uracil bases.

Terminators for use in accordance with the present disclosure include any terminator of transcription described herein or known to one of ordinary skill in the art. Non-limiting examples of terminators include the termination sequences of genes, such as, for example, the bovine growth hormone terminator, the E. coli ribosomal RNA T1T2 terminators, rrnBT1 and rrnBT2, the human preproparathyroid PTH terminator and viral termination sequences and their derivatives such as, for example, the T0 terminator, the TE terminator, Lambda T1, T7, TT7, T7U, TT3 terminators, and other terminator sequences found and/or used in bacterial systems. In some embodiments, the termination signal may be a sequence that cannot be transcribed or translated, such as those resulting from a sequence truncation.

In some embodiments, a terminator comprises two or more individual and/or distinct terminator sequences or combinations thereof. In some embodiments, a terminator comprises an rrnBT1, rrnBT2, TT7, T7U, TT3, and/or PTH terminator sequence. In some embodiments, a terminator is as described in Table 2. In some embodiments, a terminator comprises any one of SEQ ID NO: 19-30.

TABLE 2
Example terminator sequences

| Terminator ID | Description | Sequence |
|---|---|---|
| Term. 10 | T7 terminator | AACCCCTTGGGGCCTCTAAACGGG |
| | | TCTTGAGGGGTTTTTTG (SEQ ID |
| | | NO: 19) |
| Term. 18 | Combination of the natural | CATCTGTTTTCTTGCAAGATCAGCT |
| | PTH and pET-T7 | GAGCAATAACTAGCATAACCCCTT |
| | terminators | GGGGCCTCTAAACGGGTCTTGAGG |
| | | GGTTTTTTGCTGAAAGGAGGAACT |
| | | ATATCCGGA (SEQ ID NO: 20) |
| Term. 26 | Combination of synthetic | CCTAGCATAACCCCGCGGGGCCTC |
| | T7U terminator, rrnBT1 | TTCGGGGGTCTCGCGGGGTTTTTTG |
| | and the pET-T7 terminator | CTGAAAGAAGCTTCAAATAAAACG |
| | | AAAGGCTCAGTCGAAAGACTGGGC |
| | | CTTTCGTTTTATCTGTTGTTTGTCG |
| | | CTGCGGCCGCACTCGAGCACCACC |
| | | ACCACCACCATTGAGATCCGGCTG |
| | | CTAACAAAGCCCGAAAGGAAGCT |
| | | GAGTTGGCTGCTGCCACCGCTGAG |
| | | CAATAACTAGCATAACCCCTTGGG |
| | | GCCTCTAAACGGGTCTTGAGGGGT |
| | | TTTTTGCTGAAAGGAGGAACTATA |
| | | TCCGGA (SEQ ID NO: 21) |
| Term. 34 | Combination of the T3 | CCTAGCATAAACCCCTTGGGTTCC |
| | terminator, rrnBT1 and the | CTCTTTAGGAGTCTGAGGGGTTTTT |
| | pET-T7 terminator | TGCTGAAAGAAGCTTCAAATAAAA |
| | | CGAAAGGCTCAGTCGAAAGACTGG |
| | | GCCTTTCGTTTTATCTGTTGTTTGT |
| | | CGCTGCGGCCGCACTCGAGCACCA |
| | | CCACCACCACCATTGAGATCCGGC |
| | | TGCTAACAAAGCCCGAAAGGAAG |
| | | CTGAGTTGGCTGCTGCCACCGCTG |
| | | AGCAATAACTAGCATAACCCCTTG |
| | | GGGCCTCTAAACGGGTCTTGAGGG |
| | | GTTTTTTGCTGAAAGGAGGAACTA |
| | | TATCCGGA (SEQ ID NO: 22) |
| Term-Quad | Combination of rrnBT2, | AAGCTTGCTTAAGCAGAAGGCCAT |
| | TT7, PTH and pET-T7 | CCTGACGGATGGCCTTTTTGCGTTT |
| | terminator | CTACCTAGCATAACCCCTTGGGGC |
| | | CTCTAAACGGGTCTTGAGGGGTTT |
| | | TTTGGCCATCTGTTTTCTTGCAAGA |
| | | TCAGCTGAGCAATAACTAGCATAA |
| | | CCCCTTGGGGCCTCTAAACGGGTC |
| | | TTGAGGGGTTTTTTG (SEQ ID NO: |
| | | 23) |
| rrBT1 | rrnBT1 terminator | TCAAATAAAACGAAAGGCTCAGTC |
| | sequence | GAAAGACTGGGCCTTTCGTTTTAT |
| | | CTGTTGTTTGTCGCTGCGGCC (SEQ |
| | | ID NO: 24) |
| TBT2 | rrBT2 terminator | TTAAGCAGAAGGCCATCCTGACGG |
| | sequence | ATGGCCTTTTTGCGTTTCTAC (SEQ |
| | | ID NO: 25) |
| TT7 | TT7 terminator sequence | CTAGCATAACCCCTTGGGGCCTCT |
| | | AAACGGGTCTTGAGGGGTTTTTTG |
| | | (SEQ ID NO: 26) |
| PET | pET-T7 terminator | GCTGAGCAATAACTAGCATAACCC |
| | sequence | CTTGGGGCCTCTAAACGGGTCTTG |
| | | AGGGGTTTTTTGCTGAAAGGAGGA |
| | | ACTATATCCGGA (SEQ ID NO: 27) |
| T7U | T7U terminator sequence | CCTAGCATAACCCCGCGGGGCCTC |
| | | TTCGGGGGTCTCGCGGGGTTTTTTG |
| | | CTGAAAGAAGCT (SEQ ID NO: 28) |
| TT3 | TT3 terminator sequence | CCTAGCATAAACCCCTTGGGTTCC |
| | | CTCTTTAGGAGTCTGAGGGGTTTTT |
| | | TGCTGAAAGAAGCT (SEQ ID NO: |
| | | 29) |
| PTH | PTH terminator sequence | CATCTGTTTT (SEQ ID NO: 30) |

### Nucleic Acid Architecture

A nucleic acid described herein may comprise any conceivable architecture. In some embodiments, the nucleic acid is linear. In some embodiments, the nucleic acid is circular. In some embodiments, the circular nucleic acid comprises an endonuclease recognition site that may allow for the circular nucleic acid to be linearized if the appropriate endonuclease cleaves the nucleic acid at said endonuclease recognition site. In some embodiments, the nucleic acid is a DNA template comprising a sequence of interest (SOI), wherein the SOI encodes an RNA product. In some embodiments, the DNA template or vector is a plasmid or a DNA construct. In some embodiments, the DNA template or vector is a plasmid, an expression cassette, a cosmid, a bacterial artificial chromosome, a yeast artificial chromosome, a bacteriophage, an adeno-associated viral vector (AAV vector), or a virus.

The present disclosure describes nucleic acid architectures that have demonstrated better effect in the production of an RNA of interest (e.g., dsRNA), than conventional architectures (e.g. hairpin). The use of nucleic acid constructs comprising two separate expression cassettes (“independent expression cassettes” architecture), each capable of expressing the sense and antisense strands of a dsRNA product, afford an advantage over other construct architectures that allow synthesis of a given dsRNA product.

In some embodiments, a nucleic acid, e.g., a vector or construct described herein, comprises two expression cassettes, as described above, that respectively allow the expression of the sense and the antisense strands of a dsRNA product which results in the dsRNA product being formed from the hybridization of the sense and antisense strands at levels higher than those from a vector carrying a single expression cassette capable of expressing a hairpin dsRNA product with the sense and antisense strands connected by a loop sequence.

For example, such a construct that allows expression of the sense and the antisense strands independently from two separate expression cassettes, results in dsRNA product titers higher than a construct carrying a single expression cassette capable of expressing transcripts where the two complementary strands of the dsRNA product are linked via a single-stranded linker or loop region to form a dsRNA hairpin structure as seen in Example 4 below.

For a dsRNA product, ‘x’ base pairs in length, in the case of a dsRNA hairpin product expressed from a single expression cassette, the successful synthesis of the hairpin requires the polymerase to successfully transcribe over a region of DNA that is (2x+l) base pairs long, where ‘l’ is the length of the “loop” connecting the sense and the antisense strands. In comparison, with constructs, where the sense and the antisense strands are expressed independently from two separate expression cassettes, each polymerase molecule only needs to transcribe a DNA that is ‘x’ base pairs in length to successfully generate full length transcripts capable of hybridizing to form the dsRNA product. When the polymerase is in excess and efficient promoter clearance is ensured via the selection of an appropriate ITS (as described herein), multiple polymerase molecules can simultaneously and independently bind the promoters of the two expression cassettes and transcribe multiple copies of the sense and the antisense transcripts from the two separate expression cassettes, generating a large amount of dsRNA product upon hybridization of the simultaneously expressed sense and antisense transcripts in the reaction. In contrast, under similar reaction conditions, a construct with a single expression cassette for the expression of a dsRNA hairpin is expected to result in lower relative yields due to the availability of only a single promoter for transcribing a single RNA molecule comprising both the sense and the antisense transcripts, which are connected via the hairpin loop. Additionally, for RNA reactions that cause high rates of abortive transcription, the increased length of the DNA template to be transcribed to get full length transcripts (‘2x+l’ compared to ‘x’) is expected to further impact yield.

As described herein, a nucleic acid, e.g., a vector or construct, comprises a promoter, optionally a promoter operably linked to an ITS, a sequence of interest (SOI), and a terminator sequence. In some embodiments, the nucleic acid comprises a T7 minimal promoter operably linked to the ITS_6 consensus ITS e.g., a sequence as in SEQ ID NO: 17. In some embodiments, the SOI to be transcribed is placed or located immediately downstream of a promoter, e.g., a class III T7 promoter. In some embodiments, a nucleic acid, e.g., a vector or construct further comprises one or more terminators or terminator sequences placed downstream of the SOI, e.g., to prevent read-through transcription beyond the SOI that would result in RNA products with additional and undesired nucleotides. In some embodiments, a nucleic acid, e.g., a vector or construct further comprises a restriction endonuclease recognition site. In some embodiments, a restriction endonuclease recognition site is placed or located downstream of the SOI, e.g., immediately downstream of the SOI, to allow linearization of the plasmid template with the corresponding restriction endonuclease before the DNA template is used in the transcription reactions. In some embodiments, a restriction endonuclease recognition site is between an SOI and a terminator. In some embodiments, a restriction endonuclease recognition site is downstream of an SOI and a terminator.

In some embodiments, a nucleic acid, e.g., a vector or construct, comprises a promoter operably linked to an ITS; a sequence of interest (SOI); a restriction endonuclease recognition site; and a terminator. In some embodiments, a nucleic acid, e.g., a vector or construct, further comprises a sequence that is a reverse complement of the ITS located downstream of the SOI, optionally between the SOI and the terminator. In some embodiments, a nucleic acid, e.g., a vector or construct, comprises one expression cassette, wherein the expression cassette minimally comprises a promoter and an SOI. In some embodiments, a nucleic acid, e.g., a vector or construct, comprises two expression cassettes, wherein each expression cassette minimally comprises a promoter and an SOI, optionally wherein each expression cassette comprises a distinct SOI. In some embodiments, a nucleic acid, e.g., a vector or construct, comprises two expression cassettes, wherein the first expression cassette comprises an SOI encoding a sense strand of a dsRNA and the second expression cassette comprises an SOI encoding an antisense strand of the dsRNA. In some embodiments, a nucleic acid, e.g., a vector or construct, comprises more than two expression cassettes, e.g., three, four, or five expression cassettes.

In some embodiments, a nucleic acid, e.g., a vector or construct, comprises two expression cassettes, wherein the first expression cassette comprises a promoter operably linked to an ITS upstream of a nucleotide sequence, e.g., SOI, encoding a sense strand of a double-stranded RNA (dsRNA), and wherein the second expression cassette comprises a promoter operably linked to an ITS upstream of a nucleotide sequence, e.g., SOI, encoding an antisense strand of the dsRNA. In some embodiments, the sense strand of the dsRNA is complementary to the antisense strand of the dsRNA. In some embodiments, the ITS is 1-15 nucleotides in length. In some embodiments, the ITS is any one of SEQ ID NO: 1-8 or 38-41.

In some embodiments, a nucleic acid, e.g., a vector or construct, comprises two expression cassettes, wherein the first expression cassette comprises a promoter operably linked to an ITS as provided in any one of SEQ ID NO: 1-8 or 38-41 upstream of a nucleotide sequence, e.g., SOI, encoding a sense strand of a double-stranded RNA (dsRNA), and wherein the second expression cassette comprises a promoter operably linked to an ITS, as provided in any one of SEQ ID NO: 1-8 or 38-41 upstream of a nucleotide sequence, e.g., SOI, encoding an antisense strand of the dsRNA. In some embodiments, the sense strand of the dsRNA is complementary to the antisense strand of the dsRNA.

In some embodiments, a nucleic acid, e.g., a vector or construct, comprises two expression cassettes, wherein the first expression cassette comprises a promoter operably linked to a nucleotide sequence, e.g., SOI, encoding a sense strand of a double-stranded RNA (dsRNA), and wherein the second expression cassette comprises a promoter operably linked to a nucleotide sequence, e.g., SOI, encoding an antisense strand of the dsRNA. In some embodiments, the sense strand of the dsRNA is complementary to the antisense strand of the dsRNA.

In some embodiments, a nucleic acid, e.g., a vector or construct, comprises two expression cassettes, wherein the first expression cassette comprises a promoter operably linked to an ITS comprising the nucleotide sequence of any one of SEQ ID NO: 1-8 or 38-41, a nucleotide sequence, e.g., an SOI, encoding a sense strand of a dsRNA, a terminator; wherein the second expression cassette comprises a promoter operably linked to an ITS comprising the nucleotide sequence of any one of SEQ ID NO: 1-8 or 38-41, a nucleotide sequence, e.g., an SOI, encoding an antisense strand of the dsRNA, and a terminator; and wherein the sense strand of the dsRNA is complementary to the antisense strand of the dsRNA. In some embodiments, the two expression cassettes are encoded by two complementary strands of the same segment of double stranded DNA to anneal to create a dsRNA molecule. In some embodiments, during transcription of the RNA, polymerase molecules begin transcription of the complementary strands from the promoters at the two ends and the polymerases move toward each other while initially traversing the two complementary strands of DNA (e.g., in a converging manner).

In some embodiments, a nucleic acid, e.g., a vector or construct, comprises two expression cassettes, wherein the first expression cassette comprises a promoter operably linked to an ITS comprising the nucleotide sequence of SEQ ID NO: 1, a nucleotide sequence, e.g., an SOI, encoding a first RNA product, a terminator; wherein the second expression cassette comprises, a promoter operably linked to an ITS comprising the nucleotide sequence of SEQ ID NO: 1, a nucleotide sequence, e.g., an SOI, encoding a second RNA product, and a terminator; and wherein the sense strand of the dsRNA is complementary to the antisense strand of the dsRNA. In some embodiments, the first expression cassette and the second expression cassette are oriented in the same direction on the given vector or DNA molecule (the same DNA strand serves as a template strand during transcription from the respective promoters of both expression cassettes). In some embodiments, the first and second expression cassettes are oriented in opposite directions in the given vector or DNA molecule (e.g., opposite strands serve as a template strand during transcription from the respective promoters of both expression cassettes). Depending on whether the two expression cassettes are oriented in the same direction or opposite directions on a given vector or DNA molecule, the RNA polymerases driving expression from the two independent promoters may move (e.g., transcribe) in the same direction on the same strand of the dsDNA molecule or in opposite directions on the two different strands of DNA.

In some embodiments, an RNA product produced using an expression cassette is flanked by an r-ITS and a reverse complement of the r-ITS (r-ITC-RC), e.g., wherein the r-ITS is at the 5′ end of the RNA product and the reverse complement of the r-ITS (r-ITS-RC) is at the 3′ end of the RNA product.

In some embodiments, a nucleic acid, e.g., a vector or construct, comprises a ‘complementary expression cassettes’ design wherein each cassette includes an ITS and optionally an ITS-RC, for the expression of a dsRNA molecule of interest. In some embodiments, an architectural design comprises ‘complementary expression cassettes’, which each include an ITS, and optionally an ITS-RC, for the expression of a dsRNA molecule of interest wherein the first expression cassette (encoding the sense strand) and the second expression cassette (encoding the antisense strand), express the sense and antisense strands of a dsRNA molecule respectively. The sense and antisense strands are encoded by two complementary strands of the same segment of double stranded DNA and anneal to create a dsRNA molecule, which includes the r-ITS (which is complementary to the ITS) and optionally the r-ITC-RC (complementary to the ITC-RC). In some embodiments, in this architecture, the sequence of interest (SOI), with or without a DNA ITS, is operably linked to two promoters on each end, with one promoter driving the expression of the sense strand of the desired dsRNA product and the other promoter driving the expression of the antisense strand of the desired dsRNA product. During transcription of the ‘complementary expression cassettes’ design, RNA polymerase molecules begin transcription of the complementary strands from the promoters at the two ends and the polymerases move toward each other while initially traversing the two complementary strands of DNA (e.g., in a converging manner).

In other embodiments, a nucleic acid, e.g., a vector or construct, architectural design comprises an ‘independent expression cassettes’ design wherein the expression cassettes for the expression of sense and antisense strands of the dsRNA molecule are encoded by completely independent segments of DNA, which may or may not be incorporated in the same plasmid, linearized template, or other DNA constructs. In some embodiments, an ‘independent expression cassettes’ design involves at least two expression cassettes that are part of the same vector or DNA molecule, wherein the first expression cassette and the second expression cassette are oriented in the same direction on the given vector or DNA molecule (e.g., the same DNA strand serves as a template strand during transcription from the respective promoters of each expression cassette). In other embodiments, an ‘independent expression cassettes’ design involves at least two expression cassettes that are part of the same vector or DNA molecule, wherein the first expression cassette and the second expression cassette are oriented in opposite directions in the given vector or DNA molecule (e.g., during transcription, the two opposite strands in the given vector or DNA molecule serve as template strands for the two expression cassettes). Depending on whether the two expression cassettes are oriented in the same direction or opposite directions on a given vector or DNA molecule, the RNA polymerases driving expression from the two independent promoters may move (e.g., transcribe) in the same direction on the same strand of the dsDNA molecule or in opposite directions on the two different strands of DNA.

In some embodiments, a nucleic acid, e.g., a vector or construct, comprises a single expression cassette, comprising a promoter operably linked to an ITS comprising the nucleotide sequence of one of SEQ ID NOs: 1-4 (or alternative versions of each with the initial G mutated to an A, as described in SEQ ID NOs: 38-41), a nucleotide sequence, e.g., an SOL encoding an RNA product (e.g., an mRNA), and a terminator and/or restriction endonuclease recognition site. In other embodiments, the initial GG is similarly mutated to AU.

In some embodiments, a nucleic acid described herein is a vector or a plasmid. In some embodiments, a vector or a plasmid requires an origin of replication, e.g., for replication of the vector or plasmid in a host. The origin of replication defines the plasmid copy number. Plasmids carrying an origin of replication from pUC18 or pUC19 are maintained at a high copy number (500-1000 copies/cell) in a host cell under specific growth conditions. In some embodiments, an origin of replication is a medium copy number origin of replication, e.g., ColE1 from pETDuet, a high copy number origin of replication, e.g., a pUC18 derived origin of replication, or a low copy number origin of replication, e.g. P15A. In some embodiments, bacterial cells, e.g., E. coli cells, carrying such plasmids can be grown to high cell densities in fermentations to yield significant quantities of plasmid DNA that can be isolated and purified.

In some embodiments, a nucleic acid, e.g., a vector or a plasmid, further comprises a selection marker that ensures maintenance during growth on selective media. In some embodiments, a selection marker is a positive selection marker, e.g., a protein or gene that confers a competitive advantage to a bacterium that contains the selection marker. In some embodiments, a selection marker is a negative selection marker, e.g., a protein or gene that inhibits the growth and/or division of a bacterium that contains the selection marker. In some embodiments, a selection marker is a mixed positive/negative selection marker, e.g., a protein or gene that can provide a competitive advantage under certain circumstances and inhibits growth and/or division under other circumstances. Examples of selectable markers include, without limitation, genes encoding proteins that increase or decrease either resistance or sensitivity to antibiotics (e.g., ampicillin resistance genes, kanamycin resistance genes, neomycin resistance genes, tetracycline resistance genes and chloramphenicol resistance genes) or other compounds. In some embodiments, a selection marker is an antibiotic-free selection marker. Other selectable markers may be used in accordance with the present disclosure.

In some embodiments, a vector or plasmid for expression of a dsRNA product, wherein the sense and the antisense strands are expressed from two distinct expression cassettes, comprises an origin of replication and a selection marker. In some embodiments, a vector or plasmid for expression of a dsRNA product, wherein the sense and the antisense strands are expressed from two distinct expression cassettes, comprises multiple copies of both expression cassettes that encodes the sense and antisense strands, e.g., 2, 3, 4, or 5 copies of each expression cassette. In some embodiments, a vector or plasmid for expression of a ssRNA product, wherein the ssRNA product is expressed from a single expression cassette, comprises an origin of replication and a selection marker. In some embodiments, a vector or plasmid for expression of a ssRNA product comprises multiple copies of the same expression cassette that encodes the ssRNA product, e.g., 2, 3, 4, or 5 copies of the same expression cassette.

In some embodiments, a restriction enzyme recognition site is recognized and/or cleaved by a restriction endonuclease. In some embodiments, a restriction endonuclease is I-SceI. Other restriction endonucleases are known and may be used in accordance with the present disclosure. Non-limiting examples include EcoRI, EcoRII, BamHI, HindIII, TaqI, NotI, HinFI, Sau3AI, PvuII, SmaI, HaeIII, HgaI, AluI, EcoRV, EcoP15I, KpnI, PstI, SacI, SalI, ScaI, SpeI, SphI, StuI, and XbaIl, FokI, AscI, AsiAI, NotI, FseI, PacI, SdaI, SgfL, SfiI, PmeI, BspQI, Esp3I, BsmBI, and SapI.

In some embodiments, a nucleic acid, e.g., a vector or construct, further comprises a sequence that is a reverse complement of the ITS located downstream of the SOI, optionally between the SOI and the terminator. In some embodiments, a reverse complement of an ITS is 100% complementary. In some embodiments, a reverse complement of an ITS is at least 75%, 80%, 85%, 90%, or 95% complementary.

In some embodiments, the nucleic acid construct (e.g. a plasmid construct) comprises a replicon, defined as the minimal unit or element that allows replication of the nucleic acid construct (e.g. plasmid DNA) in the host microbial cell. In some embodiments, the replicon includes the origin of replication (ori) at which the replication of the nucleic acid construct (e.g. plasmid DNA) is initiated and additional elements that control the replication of the nucleic acid construct (e.g. a plasmid) and its copy number in the host cell. In embodiments where the nucleic acid construct is a plasmid DNA construct, the replication of plasmid DNA is initiated at the ori by the host's DNA replication machinery. Some non-limiting examples of replicons include those that allow replication of plasmids in bacterial hosts (e.g. E. coli) such as replicons found in the ColE1 plasmid, the pBR322 plasmid (pMB1 origin of replication), the pUC18 and pUC19 plasmids (carrying the pUC replicon, a derivative of the pMB1 replicon), the R6K plasmid, the p15A plasmid, the pSC101 plasmid etc. Different replicons result in different copy numbers and yields for plasmid in a given host. For example, the ColE1 and pMB1 origins typically allow maintenance of about 15-20 copies of plasmid molecules in each cell, while the deletion of the rop gene and two point mutations in the pMB1 origin result in the temperature-inducible amplification of copy number to 500-1000 copies per cell for plasmids carrying the pUC replicon as found in the pUC18- or pUC19-derived plasmids. Additionally, plasmids used in eukaryotic microbial cells (e.g. yeast) carry an ‘Autonomous Replication Sequence (ARS)’ as a replicon, where replication is initiated. In some embodiments, the replicon minimally consists of an origin of replication.

### Kits

Some aspects of the present disclosure provide kits. The kits may comprise, for example, an engineered nucleic acid or construct as described herein, a polymerase, nucleoside triphosphates and/or nucleoside monophosphates.

The kits described herein may include one or more containers housing components and optionally, instructions of uses. Kits for research purposes may contain the components in appropriate concentrations or quantities for running various experiments. Any of the kits described herein may further comprise components needed for performing any methods described herein.

Each components of the kits, where applicable, may be provided in liquid form (e.g., in solution), or in solid form, (e.g., a dry powder). In certain cases, some of the components may be lyophilized, reconstituted, or processed (e.g., to an active form), for example, by the addition of a suitable solvent or other species (for example, water or certain organic solvents), which may or may not be provided with the kit.

In some embodiments, the kits include instructions and/or promotion for use of the components provided. Instructions can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc.

The kits may contain any one or more of the components described herein in one or more containers. The components may be prepared sterilely, packaged in syringe and shipped refrigerated. Alternatively it may be housed in a vial or other container for storage. A second container may have other components prepared sterilely. Alternatively the kits may include the active agents premixed and shipped in a vial, tube, or other container.

The kits may also include other components, depending on the specific application, for example, containers, cell media, salts, buffers, reagents, syringes, needles, disposable gloves, etc.

### EXAMPLES

### Example 1: Production of dsRNA from Linear DNA Templates with Different ITS Variants Using In Vitro Transcription (IVT) Reactions

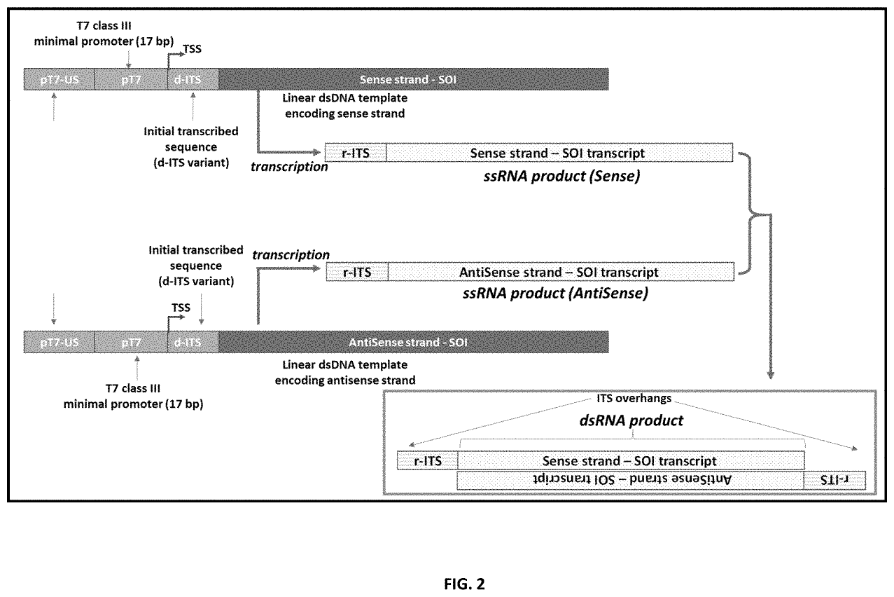

GS1, a 601-base pair dsRNA, was produced in in vitro transcription (IVT) reactions in buffer using linear DNA templates comprising expression cassettes comprising a promoter operably linked to different ITS variants (as shown in Table 3) placed immediately upstream of the sequences-of-interest (GS1 sense and GS1 antisense strands) to be expressed. Each GS1 strand synthesized in the IVT reactions comprises a 5′ single-stranded r-ITS overhang corresponding to the different ITS variants. DNA templates lacking a specific ITS (ITS_none) were used as a control in the experiment. Note that, for each ITS variant and the control, two DNA templates were utilized, one of which comprised a sequence of interest (SOI) encoding the sense strand of GS1 and the other of which comprised a SOI encoding the antisense strand of GS1. FIG. 2 shows the general architecture of the linear DNA templates with expression cassettes and the expected dsRNA product (with 5′ single stranded r-ITS overhangs).

TABLE 3
ITS variants for use in Example 1

| | ITS | |
|---|---|---|
| | sequence | Promoter* + ITS sequence |
| ITS_ | None | TCGATTCGAACTTCTGATAGACT |
| None | | TCGAAATTAATACGACTCACTA |
| | | TA (SEQ ID NO: 31) |
| ITS_6 | GGGAGA | TCGATTCGAACTTCTGATAGACT |
| | (SEQ ID | TCGAAATTAATACGACTCACTA |
| | NO: 8) | TAGGGAGA (SEQ ID NO: 32) |
| pT7- | GGGAGACC | TCGATTCGAACTTCTGATAGACT |
| g10 | ACAACGT | TCGAAATTAATACGACTCACTA |
| | (SEQ ID | TAGGGAGACCACAACGT (SEQ ID |
| | NO: 5) | NO: 33) |
| pT7-g5 | GGGAGACC | TCGATTCGAACTTCTGATAGACT |
| | GGAATT | TCGAAATTAATACGACTCACTA |
| | (SEQ ID | TAGGGAGACCGGAATT (SEQ ID |
| | NO: 6) | NO: 34) |
| GL- | GGGAGAC | TCGATTCGAACTTCTGATAGACT |
| Hybrid_ | CGGGAATT | TCGAAATTAATACGACTCACTA |
| A9 | (SEQ ID | TAGGGAGACCGGGAATT (SEQ ID |
| | NO: 1) | NO: 35) |

*The promoter operably linked to each ITS in these templates was the 47 bp extended T7 promoter (i.e. SEQ ID NO. 18), comprising the 17 bp minimal class III T7 promoter with 30 bp of DNA sequence native to the T7 bacteriophage genome, naturally found upstream of it.
Highlighted in bold is the minimal T7 class III promoter, followed by the specific ITS variants in italics.

Each IVT reaction comprised 45 mM magnesium sulfate, 2 mM spermidine, 4 mM of each of the four canonical NTPs (New England Biolabs, Ipswich, Mass.), 0.1 mg/mL of a recombinant thermostable mutant T7 RNA polymerase, 0.04 U/μL of thermostable inorganic pyrophosphatase (TIPP) (New England Biolabs, Ipswich, Mass.), and 20 ng/μL each of the two DNA templates (encoding the sense and the antisense strands of GS1 respectively). Reactions were run at 48° C. for 2 hours. After 2 hours, RNA products were isolated and quantitated using reverse phase ion pair (RP-IP) chromatography as described below.

For RP-IP HPLC analysis, samples were taken from each reaction and total RNA was extracted from the samples using solid phase extraction. Extracted dsRNA samples were analyzed by RP-IP-HPLC on an Agilent 1100 series HPLC system. HPLC analyses were performed using a DNASep® Cartridge (4.6×50 mm, ADS Biotec, PN: DNA-99-3501) held at a temperature of 50° C., with a gradient separation as shown in Table 4 (flow rate of 0.85 mL/minute). Signal was measured as absorbance at 260 nm.

TABLE 4
RP-IP HPLC gradient

| | % Mobile Phase A | % Mobile Phase B |
|---|---|---|
| Time | (0.1 M triethylammonium | (0.1 M triethylammonium |
| (min) | acetate in water) | acetate in 25% acetonitrile) |
| 0.0 | 80% | 20% |
| 1.5 | 50% | 50% |
| 7 | 32% | 68% |
| 7.01 | 0% | 100% |
| 7.5 | 0% | 100% |
| 7.51 | 80% | 20% |
| 11.5 | 80% | 20% |

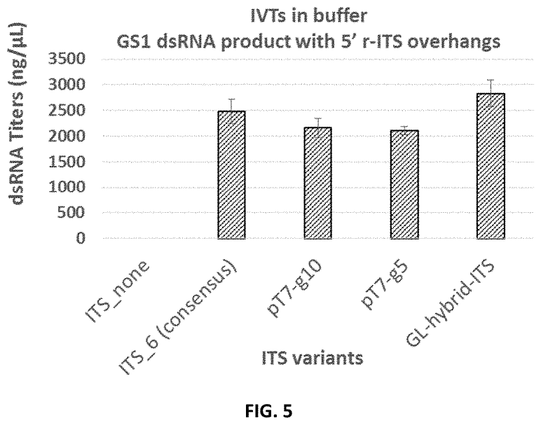

As shown in FIG. 5, all of the templates comprising one of the ITS from Table 3 (ITS_6; pT7-g10; pT7-g5; and GL-Hybrid_A9) provided a high yield of dsRNA products in IVT reactions with expression levels between 2000-2800 ng/μL. The GL-Hybrid_A9 ITS resulted in the highest expression levels of GS1 dsRNA with approximately 2800 ng/μL of synthesized GS1. As expected, the DNA template lacking a specific ITS (ITS_none) did not produce detectable expression of dsRNA product; this DNA template lacked a terminal ‘G’ at the end of the minimal class III T7 promoter that is known to be critical for transcription.

### Example 2: Production of dsRNA from Linear DNA Templates with Different ITS Variants Using Cell-Free RNA Synthesis Reactions

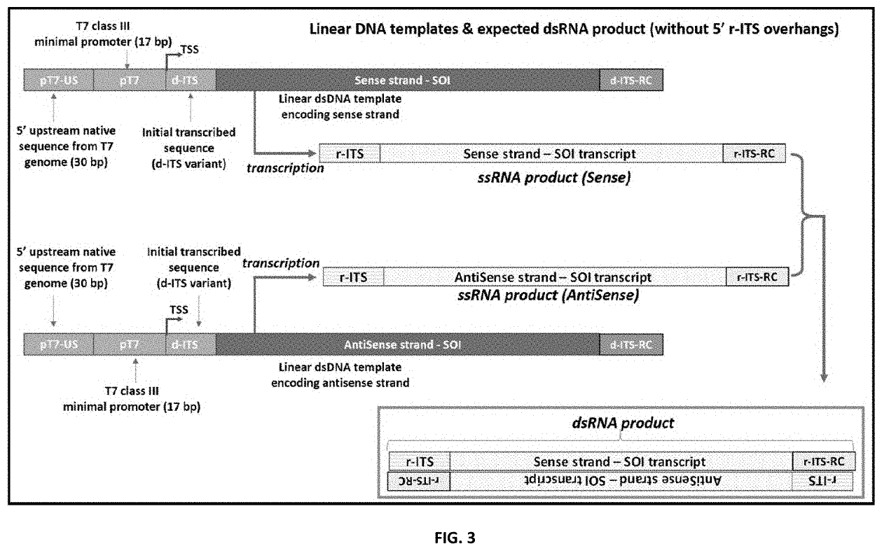

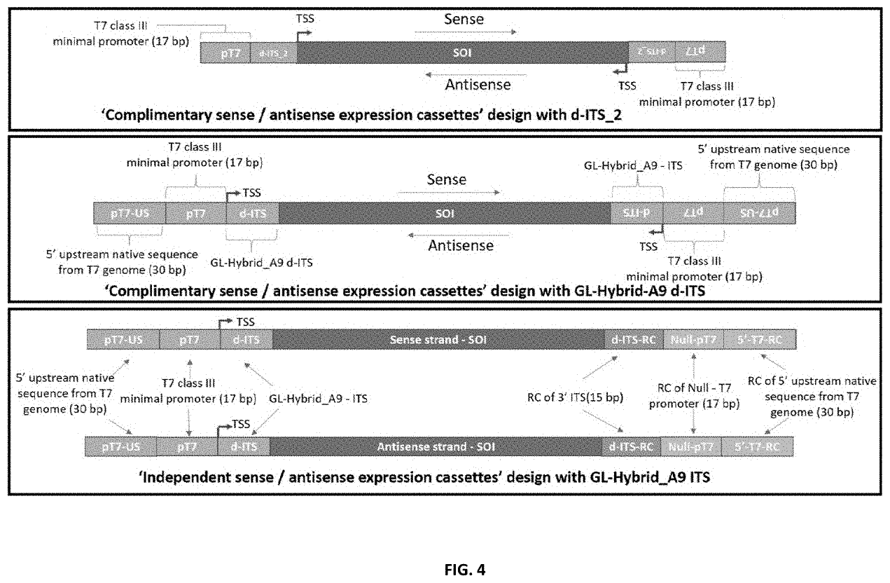

GS1 dsRNA products (with 5′ overhangs) were produced in cell-free reactions using linear DNA templates comprising expression cassettes comprising nucleotide sequences comprising a promoter operably linked to different ITS variants (as shown in Table 3), placed upstream of the SOI (GS1 sense and GS1 antisense strands) to be expressed. DNA templates lacking an ITS (ITS_none) were used as a control in the experiment. As in Example 1, for each ITS variant and the control, two DNA templates as shown in FIG. 2 were utilized, one of which comprised an SOI encoding the sense strand of GS1 and the other of which comprised an SOI encoding the antisense strand of GS1. Additionally, GS1 dsRNA products without single stranded overhangs, as shown in FIG. 3, were also produced using DNA templates according to FIG. 3 that further comprised reverse complements of their corresponding ITSes downstream of the SOI.

Yeast RNA powder, obtained from commercial sources, was dissolved in water at 56 g/L and depolymerized using 1.2 g/L P1 nuclease at 70° C., pH 5.5 for 1 hour in the presence of 0.05 mM zinc chloride. The resulting depolymerized material was clarified by centrifugation and filtered using a 10 kDa MWCO filter. The resulting stream contained 5′ nucleotide monophosphates (NMPs) at a total concentration of about 90 to 100 mM (about 20 to 25 mM of each of AMP, CMP, GMP, and UMP).

E. coli BL21(DE3) derivatives carrying pBAD24-derived vectors encoding individual kinase enzymes (TthCmk, PfPyrH, TmGmk, AaNdk, and DgPPK2) were cultivated in fermentations with Korz media supplemented with 50 mg/L carbenicillin using standard techniques and simple fed-batch technique for high cell density cultivation of Escherichia coli. Protein expression was then induced by adding L-arabinose. After harvesting the bacteria, lysates were prepared in 60 mM phosphate buffer using high-pressure homogenization, resulting in mixtures of approximately 40 g/L total protein.

E. coli BL21(DE3) derivatives carrying pBAD24-derived vectors encoding thermostable T7 RNA polymerase enzymes were cultivated, protein expression was induced using L-arabinose, and lysates were prepared as described above. Polymerase enzymes were partially purified using two steps of ammonium sulfate fractionation.

In order to assemble the cell-free reactions, lysates containing kinase enzymes were combined in equal proportion, diluted to a final total protein concentration of 2 g/L and mixed with reaction additives (45 mM magnesium sulfate and 13 mM sodium hexametaphosphate). Lysates were incubated at 70° C. for 15 minutes to inactivate other enzymatic activities while preserving the activities of the overexpressed kinases. Additionally, the yeast-derived NMPs, at a concentration of about 4 mM each, and 0.1 mg/mL of a recombinant thermostable mutant T7 RNA polymerase were added, along with 10 ng/μL each of the two linear DNA templates (expressing the sense and antisense strands of the dsRNA product respectively). Cell-free reactions were incubated at 48° C. for 2 hours and RNA products were isolated and quantitated using RP-IP HPLC as described in Example 1.

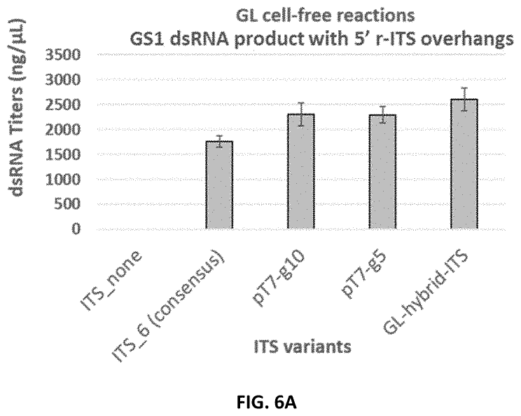

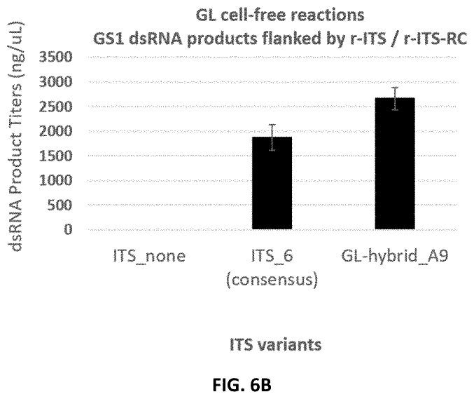

As shown in FIG. 6A, all of the templates comprising an ITS produced at least ˜1800 ng/μL levels of RNA, with GL-Hybrid_A9 ITS resulting in the highest expression level (˜2500 ng/μL) of GS1 dsRNA (with 5′ r-ITS overhangs). The ITS_None variant as expected, again showed no product synthesis while the ITS_6 variant, naturally found preceding the φ6.5, φ10 and φ13 genes in the T7 genome resulted in titers of ˜1800 ng/μL.

As shown in FIG. 6B, templates encoding reverse complements of the ITSes downstream of the SOI performed comparably to those without the reverse complements of the ITSes. The template comprising GL-Hybrid_A9 ITS showed ˜2500 ng/μL dsRNA.

Collectively these results suggest that the GL-Hybrid_A9 ITS variants produce higher levels of RNA product than the naturally occurring consensus ITS_6 variant, found preceding the φ6.5, φ10 and φ13 genes in the T7 genome.

### Example 3: Production of dsRNA from Linear DNA Templates with the GL-Hybrid_A9 ITS for Five Different SOIs

The production of five different dsRNA products (GLSeq-A, GLSeq-B, GLSeq-C, GLSeq-D & GLSeq-E) was assessed to demonstrate the benefits in RNA synthesis achieved with the use of the GL-Hybrid_A9 ITS in DNA templates. As controls in this study, templates with the ITS_2 ITS, which introduces two Gs at the end of the minimal class III T7 promoter known to be critical for transcription, were used. The T7 class III minimal promoter operably linked to ITS_2 comprises the sequence: TAATACGACTCACTATAGG (SEQ ID NO: 36). Additionally, the expression of the sense and antisense strands of a dsRNA product from two independent expression cassettes encoded on separate segments of double stranded DNA (‘independent expression cassettes’ architecture design) relative to expression from expression cassettes encoded by two complementary strands of the same segment of DNA (‘complementary expression cassettes’ design) was determined for all five SOIs using three different DNA template architectures as shown in FIG. 4: (a) ‘complementary expression cassettes’ design with ITS_2; (b) ‘complementary expression cassettes’ design with the GL-Hybrid_A9 ITS; and (c) ‘independent expression cassettes’ design with the GL_Hybrid_A9 ITS.

Note that for the two ‘complementary expression cassettes’ designs (a) and (b), the two expression cassettes, encoding the sense and antisense strands of the dsRNA products were on the two complementary strands of a double stranded DNA template with the promoters oriented towards each other, such that the sense and antisense strands of the dsRNA product were transcribed by T7RNAP transcribing the two complementary strands in opposite directions. The ‘independent expression cassettes’ design (c) is similar to the templates described in Examples 1 and 2 and involves expression of the sense and antisense strands from two expression cassettes from two independent linear DNA templates. Templates with architecture (a) employed the T7 minimal class III T7 promoter (SEQ ID NO: 9) while templates with architectures (b) & (c) employed the extended 47 base pair T7 promoter (SEQ ID NO: 18). Thus, for a given product, comparison of dsRNA production with template architectures (a) and (b) shows the effect of using the GL-Hybrid_A9 ITS with the extended T7 promoter compared to the ITS_2 variant with the minimal class III T7 promoter. Comparison of (b) & (c) shows the effect of employing the ‘independent expression cassettes’ architecture compared to the ‘complementary expression cassettes’ architecture.

Each nucleic acid architecture in combination with different SOIs (ranging up to 600 bp in length) encoding each dsRNA product (GLSeq-A, GLSeq-B GLSeq-C, GLSeq-D, and GLSeq-E) was evaluated in transcription reactions using NTPs. Cell-free reactions were similar to those described in Example 2, comprising 15 mM magnesium sulfate, 2 mM spermidine, 3.5 mM sodium hexametaphosphate and a mix of lysates containing the five kinases at a concentration of 10 mg/mL of total protein. However, unlike in Example 2, NTPs at a concentration of 4 mM of each NTP were used in place of yeast-derived NMPs. For each SOI, 50 ng/μL of either the ‘complementary expression cassettes design with ITS_2’ or ‘complementary expression cassettes design with GL_Hybrid_A9 ITS’ DNA templates were added to the reaction mix. For each SOI, in the ‘independent expression cassettes design with GL-Hybrid_A9 ITS’ design, two DNA templates (one comprising an SOI encoding the sense strand; the other comprising an SOI encoding the antisense strand) were added to the reaction at a concentration of 50 ng/μL of each. Finally, to initiate transcription, a recombinant thermostable T7 RNA polymerase mutant was added at a concentration of 0.3 mg/mL. Reactions were incubated at 48° C. for 2 hours before RNA products were isolated and analyzed via RP-IP HPLC as described in Example 1.

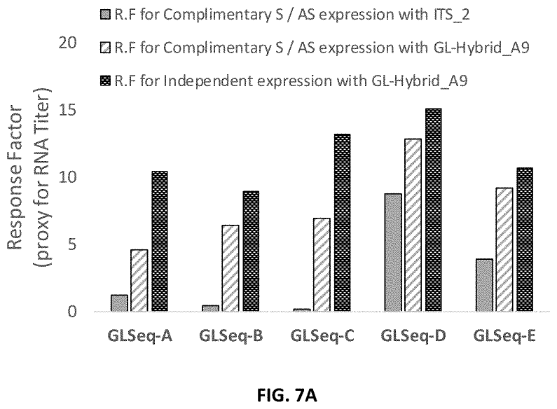

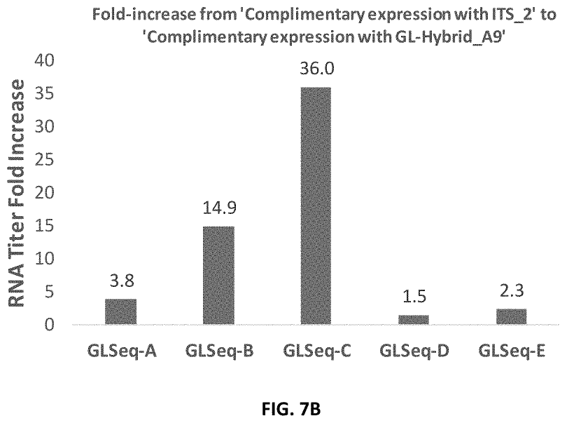

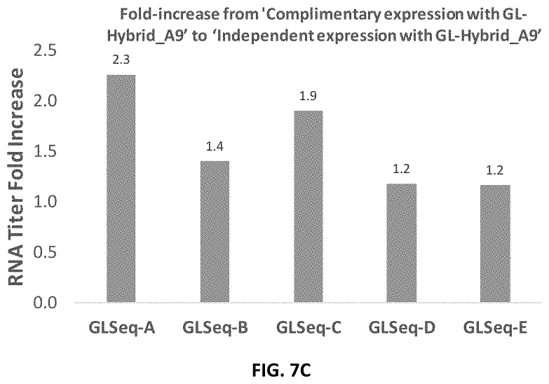

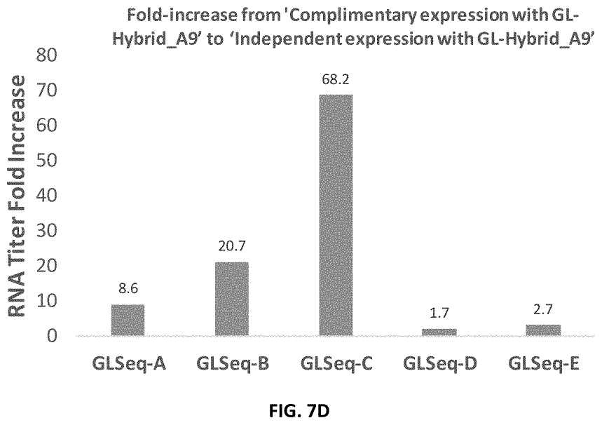

Production of dsRNA product for each SOI was increased when being transcribed from a DNA template comprising the ‘complementary expression cassettes with the GL-Hybrid_A9’ design, relative to the template comprising the ‘complementary expression cassettes with ITS_2’ (FIG. 7B). Incorporation of the GL-Hybrid_A9 ITS into DNA templates consistently resulted in an increase in the dsRNA product titers for each of the SOIs, as had been shown with GS1 (Examples 1-3). Notably, the extent of improvement was specific to the SOI (ranging between roughly 2 to 36-fold). There was a large variability in the dsRNA titers achieved for the five different SOIs in expression from the DNA templates with the ‘complementary expression cassettes with ITS_2’ design (FIG. 7A). Conversely, the incorporation of the GL-Hybrid_A9, in either the ‘complementary expression cassettes’ or ‘independent expression cassettes’ designs, resulted in consistently similar dsRNA titers for all five SOIs.

Production of each RNA product was increased when being transcribed from a DNA template comprising the ‘independent expression cassettes with GL-Hybrid_A9’ design, relative to the template comprising the ‘complementary expression cassettes with GL-Hybrid_A9’ (FIG. 7C). For the templates employing the GL-Hybrid_A9 ITS, all five dsRNA SOI products were produced at higher expression levels when transcribing a template with a ‘independent expression cassettes’ design (1.2-2.3 fold increase in relative RNA titers) compared to the ‘complementary expression cassettes’ design.

The overall effects of incorporating the GL-Hybrid_A9 ITS into a DNA template upstream of an SOI and expressing the sense and antisense strands of a dsRNA from an ‘independent expression cassettes’ design are provided in FIG. 7D. Total fold improvement in RNA titers when transitioning from a complementary expression cassettes design with ITS_2 to an independent expression cassettes design with a GL-Hybrid_A9 ITS ranges between roughly two to seventy fold.

### Example 4: Comparison of Production of GLSeq-A dsRNA Variants from Plasmid DNA Templates Employing an ‘Independent Expression Cassettes’ Architecture & a Hairpin Architecture

The production of GLSeq-A dsRNA using two different plasmid DNA templates was compared using cell-free and IVT reactions.

Plasmid construct-1 comprised two separate expression cassettes for the expression of two strands of the GLSeq-A dsRNA molecule, with each expression cassette comprising the extended 47 bp T7 promoter (SEQ ID NO:18) operably linked to the GL-Hybrid_A9 ITS and located upstream of a DNA template (SOI) encoding either the sense or antisense strand of the GLSeq-A and a downstream terminator comprising Terminator 18 (SEQ ID NO: 20).

Plasmid construct-2 comprised a single expression cassette for the expression of a hairpin variant of the GLSeq-A dsRNA molecule wherein the sense and antisense strands were connected by a single stranded linker loop. The single expression cassette comprised the extended 47 bp T7 promoter operably linked to the GL-Hybrid_A9 ITS and located upstream of a DNA template (SOI) encoding the antisense strand of GLSeq-A, a DNA sequence encoding the single stranded loop region of the hairpin, a DNA template (SOI) encoding the sense strand of GLSeq-A, and a downstream terminator comprising Terminator 18 (SEQ ID NO: 20).

Each plasmid construct was evaluated in cell-free reactions, similar to those described in Example 3, using either NTPs or yeast derived NMPs as a substrate. In comparing dsRNA production using each of the two plasmid constructs, the concentration of the plasmids in the respective reactions were adjusted to achieve roughly the same number of promoters driving transcription. Thus, plasmid construct-1 was used at a concentration of ˜60 ng/μL while plasmid construct-2 was used at a concentration of ˜100 ng/μL.

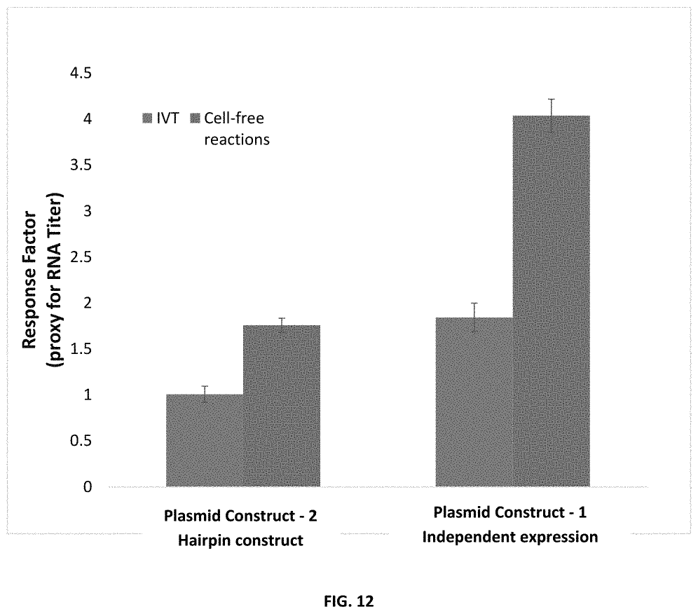

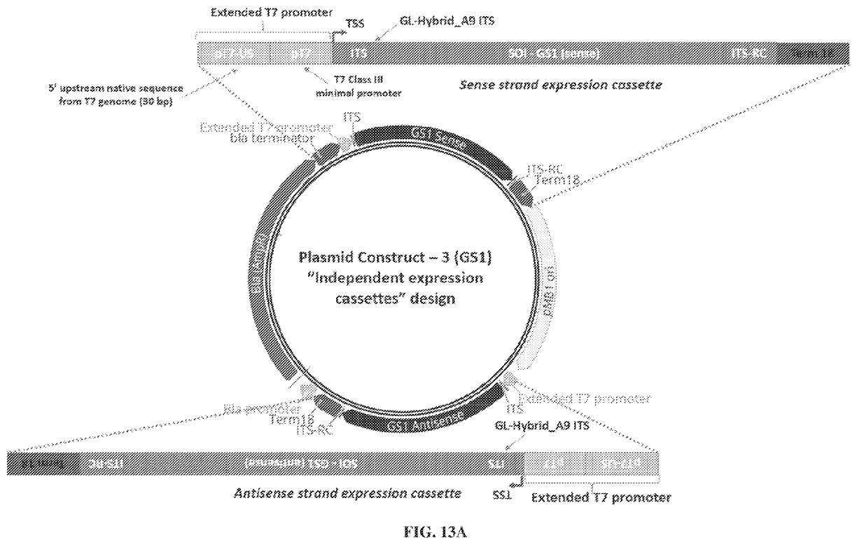

As shown in FIG. 12, the expression of the GLSeq-A dsRNA product from two independent expression cassettes encoding the sense and the antisense strands resulted in about a two-fold improvement in the dsRNA titers in both IVT and cell-free reactions compared to expression as a hairpin GLSeq-A dsRNA product from a single expression cassette.

### Example 5: Evaluation of Read-Through & Termination Efficiency with Different Terminator Constructs

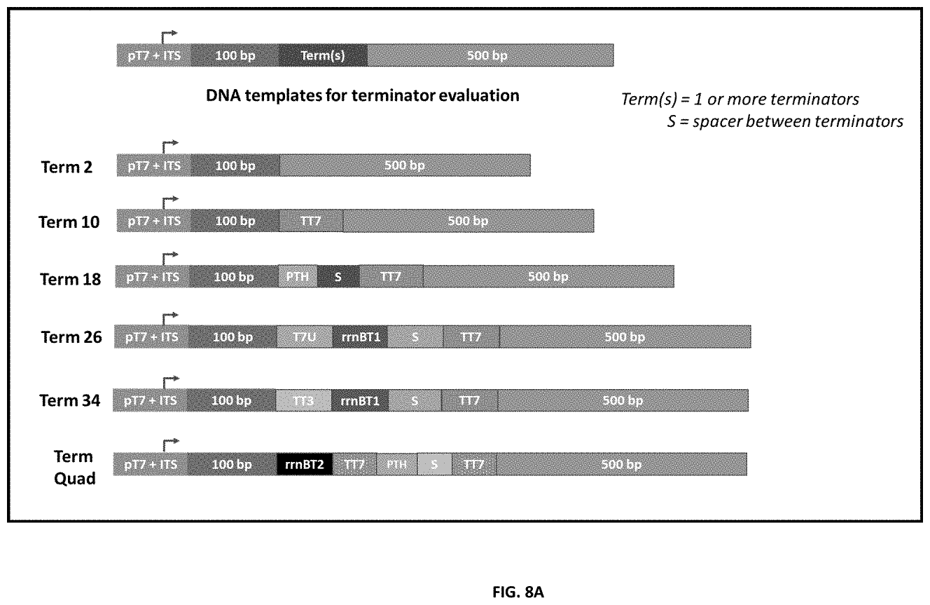

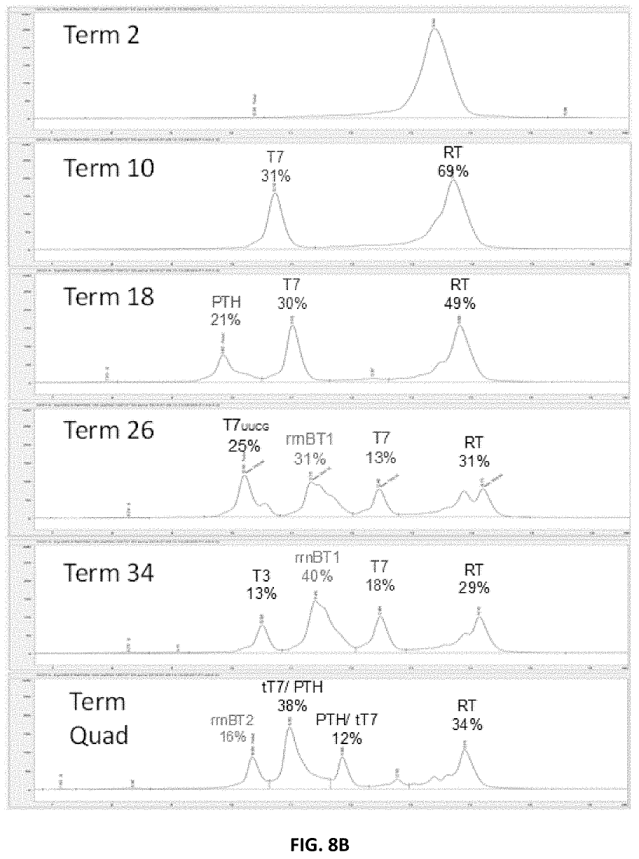

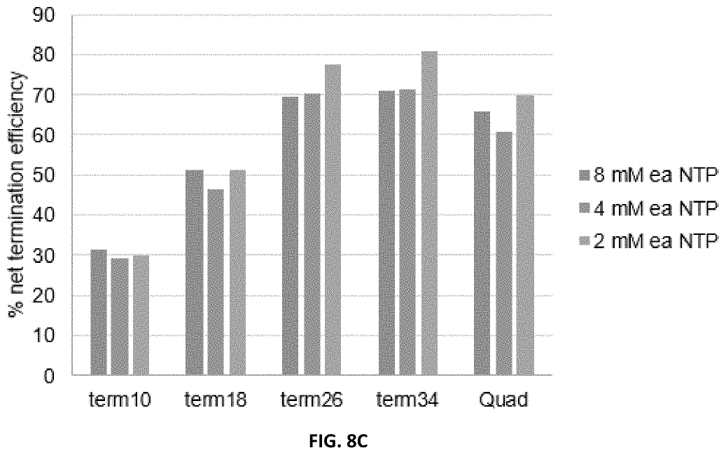

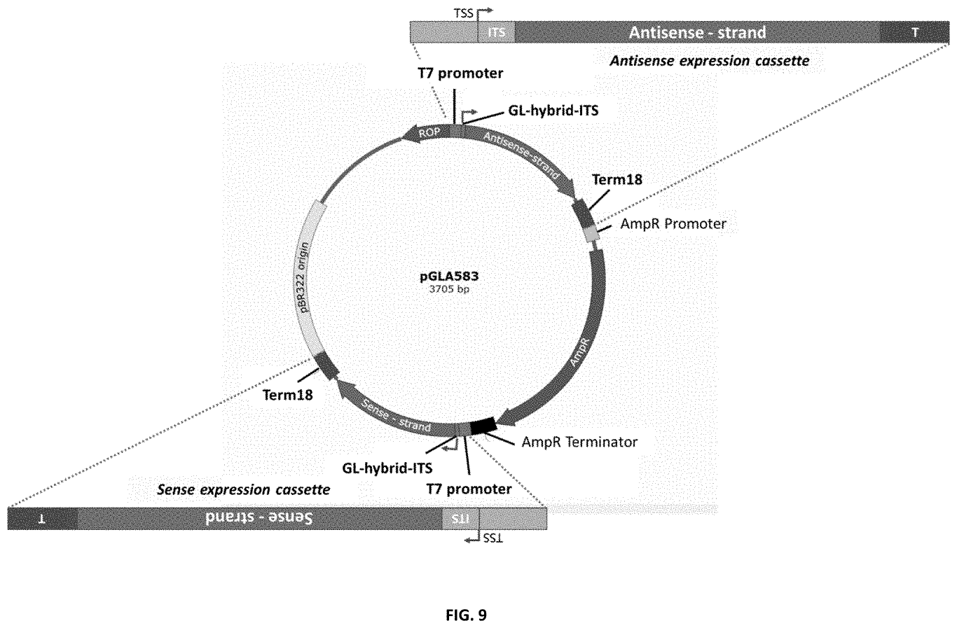

Combinations of terminators described in Table 2 were evaluated in terms of synthesis of ssRNA products driven by a T7 promoter operably linked to the GL-Hybrid_A9 ITS in in vitro transcription reactions. Terminator(s) were placed downstream of a 115 bp SOI (where transcription of the SOIs was driven by the 47 bp extended T7 promoter operably linked to the 15 bp GL-Hybrid_A9 ITS). Thus termination by the first terminator in the combination of terminators being tested would result in a product that is roughly 115 nucleotides in length. Read-through is expected to result in a distribution of products ranging in length from 115 to 912 nucleotides, depending on specific combination of terminators employed and the location of termination. The exact constructs used in this Example are presented in FIG. 8A.

These different variants were evaluated in in vitro transcription reactions using 5′ nucleotide triphosphates as a substrate. The reaction mix was composed of 45 mM magnesium sulfate, 2 mM spermidine, 0.5, 2, 4 or 8 mM of each of the four NTPs (New England Biolabs, Ipswich, MA), 0.3 mg/mL of a thermostable T7 RNA polymerase mutant. 50 ng/μL of DNA template (containing a particular terminator or combination of terminators) was added to each of the reactions. Reactions were run at 48° C. for 2 hours and subsequent RNA products were isolated and quantitated using RP-IP HPLC.

For each terminator or combinations of terminators, the different ssRNA products synthesized in the IVT reactions were separated using RP-IP HPLC, as described in Example 1. FIG. 8B shows the chromatograms for the different terminator constructs. Percentage Read-Through (% RT) was calculated as the peak area for the read through product peak (the final peak in the chromatogram observed as a result of polymerase read-through all of the terminators) expressed as a percentage of the total peak area under the curve (Table 5). Term 26, Term 34 and Term-Quad show the highest degree of termination and the lowest % RT values. Notably, all terminators performed at comparable levels in reactions comprising 2 mM, 4 mM, or 8 mM NTPs (FIG. 8C)

TABLE 5
% RT for the different terminator constructs

| | | | Termina- |
|---|---|---|---|
| Term | | | tion Effi- |
| ID | Description | % RT | ciency (%) |
| Term. 2 | No terminator control | 100% | 0% |
| Term. | T7 terminator | 69% | 31% |
| 10 | | | |
| Term. | Combination of the natural PTH and | 49% | 51% |
| 18 | T7 terminators | | |
| Term. | Combination of synthetic T7 terminator, | 31% | 69% |
| 26 | rrnBT1 and the natural T7 terminator | | |
| Term. | Combination of the T3 terminator, | 29% | 71% |
| 34 | rrnBT1 and the natural T7 terminator | | |
| Term- | Combination of rrnBT2, T7, PTH and | 34% | 66% |
| Quad | T7 terminator | | |

100% efficient termination was expected to result in an ssRNA product that is about 115 nucleotides in length. Read-through was expected to result in a distribution of products between 115-912 nucleotides in length, depending on the site of termination and the specific combination of terminators used. For example, with the dual terminator (Term 18) which carries a combination of the PTH and T7 terminators, termination at or within the PTH terminator is expected to result in a product that is about 115-125 nucleotides long (given that the PTH terminator is placed immediately downstream of the SOI and is itself about 10 nucleotides long). However, if the PTH terminator is unsuccessful in termination and termination is observed to occur at the T7 terminator, this would result in an ssRNA product anywhere between 137 to 221 nucleotides in size (depending on where within the 84 bp long T7 terminator, termination occurs). Finally, if T7 RNA polymerase reads through both the PTH and T7 terminators, then the resulting ssRNA product would be expected to be 721 nucleotides long.

The ssRNA products obtained from reactions with DNA templates demonstrated that all of Term 18, Term 26, Term 34 and Term-Quad showed % RT of less than 50%, providing an overall termination efficiency greater than 50%. In particular, Term 26, Term 34 and Term-Quad provided high termination efficiencies (65-70%).

### Example 6: Production of dsRNA from Plasmid Constructs (pGLA583 & pGLA584) and Linear DNA Templates with the GL-Hybrid_A9 ITS

Plasmid DNA templates for expression of GS4 dsRNA product were designed. GS4 is a 554 bp long dsRNA molecule that comprises a 524 base pair RNA product of interest, encoding part of the green fluorescence protein (GFP) gene flanked by the GL-Hybrid_A9 r-ITS and the reverse complement of GL-Hybrid_A9 (r-ITS-RC). Plasmid DNA templates (pGLA583 and pGLA584) with the ‘independent expression cassettes’ architecture were designed to express the sense and the antisense strands of GS4 from two separate expression cassettes (See, FIG. 4). Each of these expression cassettes comprised an extended T7 promoter (SEQ ID NO: 18) operably linked to the GL-Hybrid_A9 ITS, an GS4 SOI (either the sense or the antisense strand), the reverse complement of the GL-Hybrid_A9 ITS (ITS-RC) and 2 terminators (Term 18 (SEQ ID NO: 20)). Both pGLA583 and pGLA584 plasmids further comprised an antibiotic resistance marker (bla gene preceded by a constitutive promoter that conferred resistance to ampicillin/carbenicillin) and an origin of replication. pGLA583 is a medium copy number plasmid with a pBR322 origin of replication derived from a pETDuet vector; pGLA584 is a high copy number plasmid with a mutated pBR322 origin of replication (FIG. 9).

Additionally, two linear DNA templates that encode expression cassettes for expression of the GS4 sense and antisense strands, flanked by sequences corresponding to the GL-Hybrid_A9 r-ITS and its reverse complement were designed.

The ability of the two plasmids and the linear templates to produce GS4 dsRNA in cell-free reactions was tested. Cell lysates containing kinase enzymes (prepared as described in Example 2) were combined in equal proportion, diluted to a final total protein concentration of 1.75 g/L and mixed with reaction additives (45 mM magnesium sulfate and 13 mM sodium hexametaphosphate). Lysates were incubated at 70° C. for 15 minutes to inactivate other enzymatic activities while preserving the activities of the overexpressed kinases. Finally, NMPs (derived by depolymerization of cellular yeast RNA) at a concentration of roughly 7-8 mM each and 0.1 mg/mL of a thermostable T7 mutant RNA polymerase were added with either (A) 10 ng/μL each of the two linear DNA templates (expressing the sense and antisense strands of the dsRNA product respectively) or (B) 120 ng/μL of the plasmid DNA templates (pGLA583 or pGLA584). Cell-free reactions were incubated at 48° C. for 2 hours and subsequent RNA products were isolated and quantitated using RP-IP HPLC.

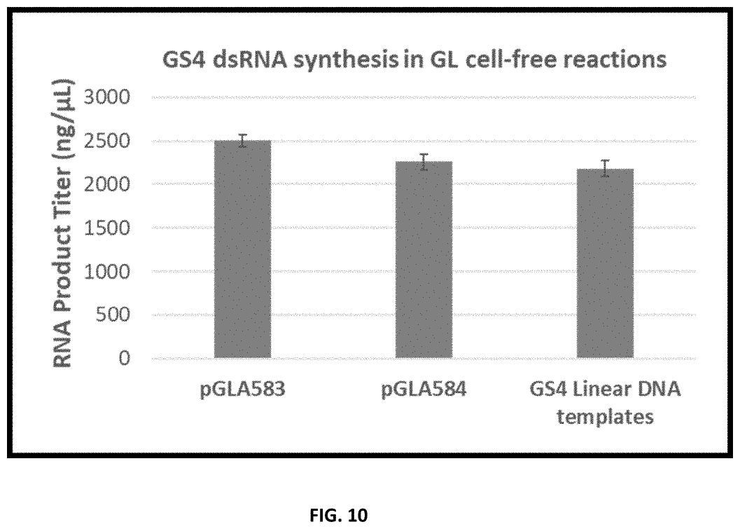

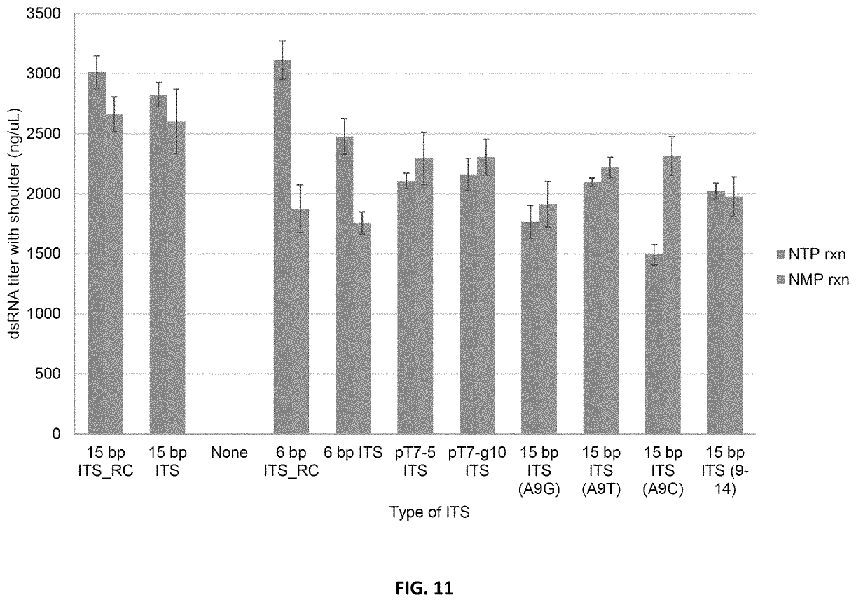

High expression levels of dsRNA product were observed from each of the DNA templates (FIG. 10). The linear DNA templates produced ˜2100 ng/μL; the two plasmid templates (pGLA583 and pGLA584) each produced higher levels, with pGLA583 producing ˜2500 ng/μL.

### Example 7: Production of Dsrna Products Using Plasmid Templates Employing the ‘Independent Expression Cassettes’ Versus ‘Complementary Expression Cassettes’ Architectures

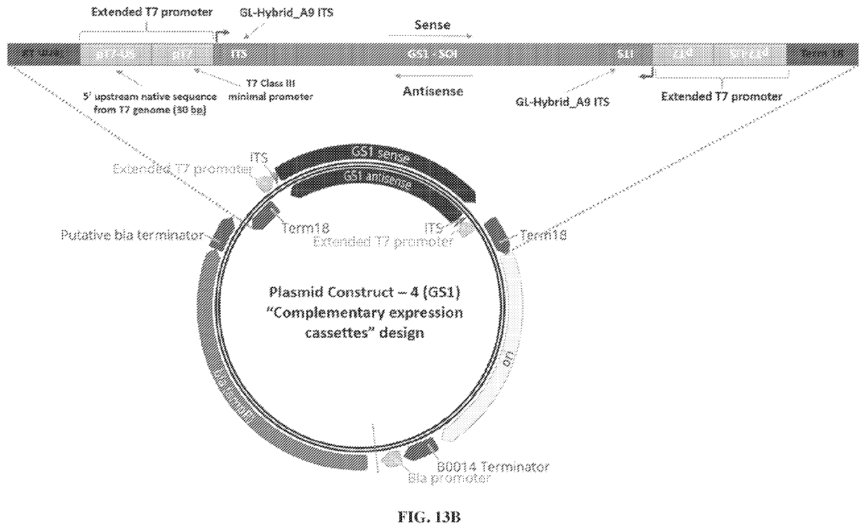

The production of two different dsRNA products (GS1 and GS4) in cell-free reactions was compared using two types of plasmid templates employing two different architectures for the production of dsRNA products (Plasmid construct-3: a plasmid DNA template employing the ‘independent expression cassettes’ architecture for expression of dsRNAs and Plasmid construct-4: a plasmid template employing the ‘complementary expression cassettes’ design for dsRNA expression). The plasmid templates employing the ‘independent expression cassettes’ architecture carried two separate expression cassettes, encoded by two separate segments of DNA for the expression of the sense and antisense strands of the dsRNA product respectively in the same plasmid, separated by an origin of replication and a selection marker as shown in FIG. 13A. The plasmid templates employing the ‘complementary expression cassettes’ architecture on the other hand carried a DNA segment where complementary strands of that DNA segment encoded expression cassettes for the expression of the sense and antisense strands of the given dsRNA product, as shown in FIG. 13B. In both architectures, the extended T7 promoter (SEQ ID NO: 18) was used in conjunction with the GL-Hybrid_A9 ITS to express each strand of the desired dsRNA products.

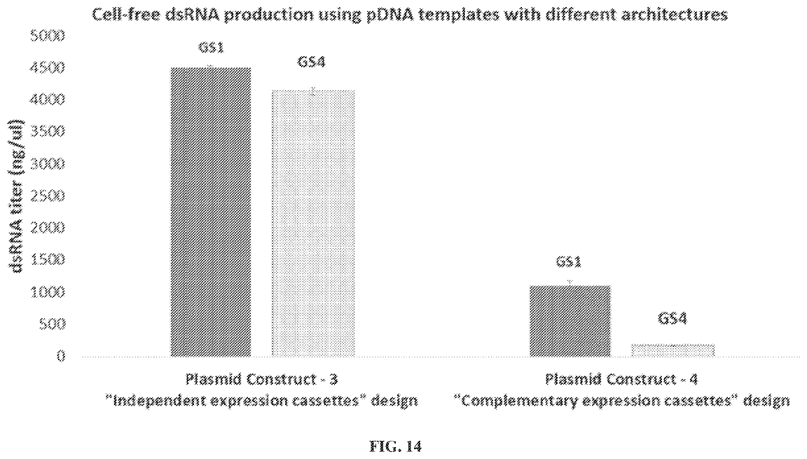

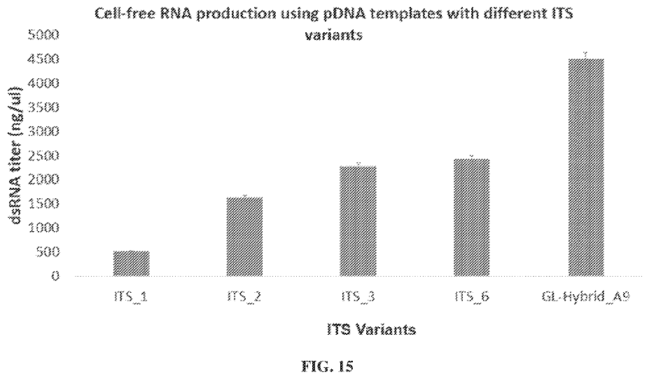

Cell-free reactions for dsRNA production were prepared as described in Example 2, using 60-100 ng/μL of plasmid DNA template employing either the ‘independent expression cassettes’ or ‘complementary expression cassettes’ architectures. As observed in FIG. 14, the independent expression of the sense and antisense strands of a dsRNA product from two separate expression cassettes from plasmids employing the ‘independent expression cassettes’ architecture (Plasmid construct-3) results in 4-20 fold higher dsRNA production compared to the expression of the same dsRNA products from DNA templates employing the ‘complementary expression cassettes’ architecture (Plasmid construct-4).

### Example 8: Production of dsRNA Products Using Plasmid Templates Employing the ‘Independent Expression Cassettes’ Architecture with Expression Cassettes Carrying Different ITSes

The production of the GS1 dsRNA product in cell-free reactions from five different plasmid DNA templates was compared, where all five plasmids employed the ‘independent expression cassettes’ architecture as shown in FIG. 13A and differed in the ITSes used in the expression cassettes for the expression of the sense and antisense strands in each plasmid. In each of the expression cassettes for the expression of the sense and antisense strands, the 47 bp extended T7 promoter (SEQ ID NO: 18) was operably linked to one of five different ITSes shown in Table 6.

TABLE 6
ITS variants for use in Example 8

| | ITS | |
|---|---|---|
| | sequence | Promoter* + ITS sequence |
| ITS_1 | G | TCGATTCGAACTTCTGATAGACT |
| | | TCGAAATTAATACGACTCACTA |
| | | TAG (SEQ ID NO: 46) |
| ITS_2 | GG | TCGATTCGAACTTCTGATAGACT |
| | | TCGAAATTAATACGACTCACTA |
| | | TAGG (SEQ ID NO: 47) |
| ITS_3 | GGG | TCGATTCGAACTTCTGATAGACT |
| | | TCGAAATTAATACGACTCACTA |
| | | TAGGG (SEQ ID NO: 48) |
| ITS_6 | GGGAGA | TCGATTCGAACTTCTGATAGACT |
| | (SEQ ID | TCGAAATTAATACGACTCACTA |
| | NO: 8) | TAGGGAGA (SEQ ID NO: 49) |
| GL- | GGGAGAC | TCGATTCGAACTTCTGATAGACT |
| Hybrid_ | CGGGAATT | TCGAAATTAATACGACTCACTA |
| A9 | (SEQ ID | TAGGGAGACCGGGAATT (SEQ ID |
| | NO: 1) | NO: 35) |

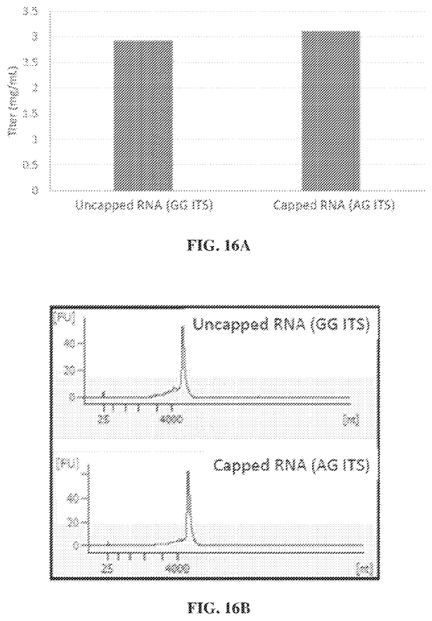

As shown in FIG. 15, the GL-Hybrid_A9 ITS resulted in a 1.9 fold improvement in dsRNA synthesis over the consensus ITS_6 naturally found in the T7 bacteriophage genome as well as other shorter ITSes.

### Example 9. ITSes Comprising an A-Start Produce Capped RNA at High Titer and Purity Using Co-Transcriptional Capping Reagents

A pair of mRNA molecules were designed and produced in cell-free reactions. Both molecules consisted of a similar sequence architecture, incorporating the 5′ ITS GL-hybrid_A9 (A start) (AGGAGACCAGGAATT (SEQ ID NO: 38)). mRNA sequences, were encoded on a pUC-19 derived plasmid template along with a T7 promoter at the 5′ end and a restriction endonuclease recognition site. Plasmids were propagated in E. coli strain DH10b, purified by Plasmid Giga Kits (Qiagen), linearized by digestion with Esp3I restriction endonucleases (New England Biolabs), and further purified by phenol-chloroform extraction. RNA synthesis reactions were performed using the cell-free production platform as described in PCT/US2020/025824. Reactions producing capped RNAs also included CleanCap AG reagent (TriLink Biotechnologies). Template DNA was removed by treatment with DNase I, then RNA was recovered by lithium chloride precipitation. Recovered RNA was quantified by UV absorbance at 260nm and analyzed for size and quality using a 2100 BioAnalyzer instrument (Agilent Technologies).

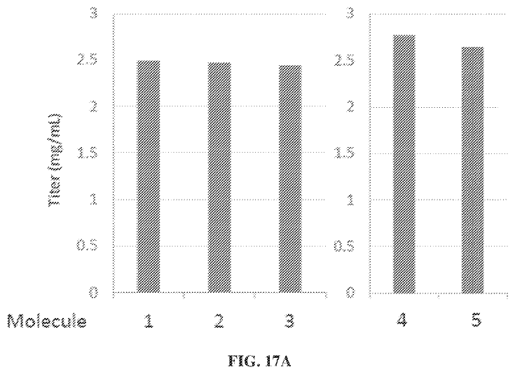

As shown in FIG. 16, cell-free reactions producing capped RNA using CleanCap AG and an AG ITS (GL-hybrid A9 (A Start), SEQ ID NO: 38) achieved similar titers as reactions producing uncapped RNA using the GG ITS (GL-hybrid_A9, SEQ ID NO: 1) (FIG. 16A). Analysis by BioAnalyzer demonstrated RNA products from both reactions were of the expected size and similar purity (FIG. 16B).

### Example 10. ITSes in mRNAs Encoding Different Proteins Results in Consistent Production Titers and Molecule Quality

A family of mRNA molecules were designed and produced in cell-free reactions. As in Example 9, sequences incorporated the 5′ ITS GL-hybrid_A9 (A start) (AGGAGACCAGGAATT (SEQ ID NO: 38)). RNA synthesis reactions were performed using the cell-free production platform as described in PCT/US2020/025824. Reactions producing capped RNAs also included 5 mM CleanCap AG reagent (TriLink Biotechnologies). Plasmids were propagated in E. coli strain DH10b, purified by Plasmid Giga Kits (Qiagen), linearized by digestion with Esp3I or B spQI restriction endonucleases (New England Biolabs), and further purified by phenol-chloroform extraction. Template DNA was removed by treatment with DNase I, then RNA was recovered by lithium chloride precipitation. Recovered RNA was quantified by UV absorbance at 260 nm and analyzed for size and quality using a Fragment Analyzer instrument (Agilent Technologies).

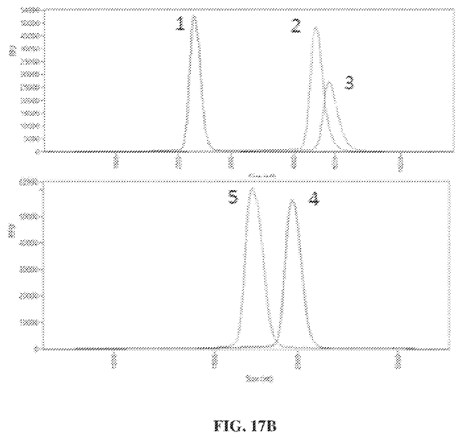

As shown in FIG. 17, cell-free reactions producing capped RNA using CleanCap AG and the AG ITS produced consistent titers across multiple open reading frame sequences (FIG. 17A). RNA products of these reactions migrated at the expected sizes. All molecules were produced with similarly high purity (FIG. 17B).

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

What is claimed is:

1. An engineered nucleic acid comprising an initial transcription sequence (ITS) comprising the nucleotide sequence of any one of SEQ ID NO: 1-4 or 38-41.

2. The engineered nucleic acid of claim 1, comprising a promoter operably linked to the ITS.

3. The engineered nucleic acid of claim 1 comprising the nucleotide sequence of any one of SEQ ID NO: 10-13 or 42-45.

4. The engineered nucleic acid of claim 1, further comprising a sequence of interest downstream of the nucleotide sequence of the ITS.

5. The engineered nucleic acid of claim 4, further comprising one or more terminator sequences downstream of the sequence of interest.

6. The engineered nucleic acid of claim 5, wherein the terminator sequence comprises a rrnBT1, rrnBT2, TT7, pET-T7, T7U, TT3, and/or PTH terminator sequence.

7. (canceled)

8. The engineered nucleic acid of claim 2, wherein the promoter is a bacteriophage T7 promoter.

9. (canceled)

10. The engineered nucleic acid of claim 1, wherein the engineered nucleic acid is double-stranded.

11. The engineered nucleic acid of claim 1, wherein the engineered nucleic acid is circular.

12. A construct comprising:
a first expression cassette comprising a promoter operably linked to an initial transcription sequence (ITS) upstream of a nucleotide sequence encoding a sense strand of a double-stranded RNA (dsRNA), wherein the initial transcription sequences comprise the nucleotide sequence of any one of SEQ ID NOs: 1-8 or 38-41; and
a second expression cassette comprising a promoter operably linked to an initial transcription sequence (ITS) upstream of a nucleotide sequence encoding an antisense strand of the dsRNA, wherein the initial transcription sequences comprise the nucleotide sequence of any one of SEQ ID NOs: 1-8 or 38-41,
wherein the sense strand of the dsRNA is complementary to the antisense strand of the dsRNA.

13-16. (canceled)

17. The construct of claim 12, wherein the initial transcription sequences comprise the nucleotide sequence of any one of SEQ ID NOs: 1-4 or 38-41.

18-19. (canceled)

20. The construct of claim 12, wherein the nucleotide sequence encoding the sense strand of the first expression cassette is flanked by the ITS and a reverse complement of the ITS and the antisense strand of the second expression cassette is flanked by the ITS and a reverse complement of the ITS.

21-26. (canceled)

27. The construct of claim 12, wherein either the promoter of the first expression cassette, the promoter of the second expression cassette, or both the promoter of the first expression cassettes and the promoter of the second expression cassettes is a bacteriophage T7 promoter.

28-32. (canceled)

33. A construct comprising:
(a) a first expression cassette comprising
a promoter operably linked to an initial transcription sequence (ITS) comprising the nucleotide sequence of any one of SEQ ID NO: 1-4 or 38-41, a nucleotide sequence encoding a sense strand of a double-stranded RNA (dsRNA), and a terminator sequence; and
(b) a second expression cassette comprising
a promoter operably linked to an ITS comprising the nucleotide sequence of any one of SEQ ID NO: 1-4 or 38-41, a nucleotide sequence encoding an antisense strand of the dsRNA, and a terminator sequence, wherein the sense strand of the dsRNA is complementary to the antisense strand of the dsRNA.

34-52. (canceled)

53. An expression cassette comprising a promoter operably linked to an initial transcription sequence (ITS) upstream of a nucleotide sequence encoding a product of interest and optionally followed by an ITS-RC and/or a restriction endonuclease site and/or two tandem terminator sequences, wherein the ITS comprises the nucleotide sequence of any one of SEQ ID NOs: 1-8 or 38-41.

54. (canceled)

55. A kit comprising:
an engineered nucleic acid comprising the nucleotide sequence of any one of SEQ ID NO: 1-4 or 38-41;
a polymerase, and further comprising nucleoside triphosphates and/or nucleoside monophosphates.

56. (canceled)
